(19) United States
(12) Patent Application Publication
(10) Pub. No.: US 2026/0294340 A1
(43) Pub. Date: Oct. 1, 2026

(54) TEMPLE MOUNTED WEARABLE DEVICE AND METHOD FOR MONITORING HEALTH ATTRIBUTES

(71) Applicant: CONTINUE LIFESCIENCES PRIVATE LIMITED, GURGAON (IN)

(72) Inventors: Ram SINGLA, GURGAON (IN); Satya PRAKASH, GURGAON (IN)

(73) Assignee: CONTINUE LIFESCIENCES PRIVATE LIMITED, GURGAON (IN)

(21) Appl. No.: 19/577,359

(22) Filed: Mar. 25, 2026

Related U.S. Application Data
(60) Provisional application No. 63/779,966, filed on Mar. 28, 2025.

(51) Int. Cl. A61B 5/00; A61B 5/026

(52) U.S. Cl. CPC A61B 5/6814; A61B 5/0261; A61B 5/721; A61B 2562/0219

(57) ABSTRACT

A temple mounted wearable device configured to monitor a set of health attributes associated with a user is disclosed. The wearable device includes a housing unit adapted to conform to a temple region of the user, with an aperture positioned over a superficial temporal artery, and a computing unit disposed within the housing unit. The computing unit comprises at least one photoplethysmography (PPG) sensor and one inertial measurement unit (IMU) sensor configured to receive physiological data from the temple region. A processor determines a motion-correction status and generates enhanced PPG data when motion correction is required. Further, blood-flow indicators are extracted from at least one of the PPG data and the enhanced PPG data to generate health metrics associated with the user. The temple mounted wearable device communicates with a user device and remote computing resources to monitor health attributes in real-time and determine a health state of the user.

### CROSS REFERENCE TO RELATED APPLICATION

The present disclosure claims benefit of U.S. provisional Application No. 63/779,966 filed Mar. 28, 2025, the disclosure of which is incorporated herein by reference in its entirety.

### TECHNICAL FIELD

The present disclosure generally relates to the field of health and fitness monitoring using wearable devices. More particularly, the present disclosure relates to a temple mounted wearable device and a method for monitoring health attributes associated with a user.

### BACKGROUND

The following description of the related art is intended to provide background information pertaining to the field of the disclosure. This section may include certain aspects of the art that may be related to various features of the present disclosure. However, it should be appreciated that this section is used only to enhance the understanding of the reader with respect to the present disclosure, and not as an admission of the prior art.

The advent of advanced sensing, processing, and wireless communication technologies has driven the development of numerous wearable devices intended to continuously monitor user activities and assess health and fitness parameters. These wearable devices are typically configured to be worn on different regions of the body, including but not limited to the fingers, wrists, arms, ears, and other peripheral locations. Such devices commonly integrate one or more sensors such as optical sensors, bioelectrical sensors, touch sensors, accelerometers, gyroscopes, and similar components along with embedded processors and communication interfaces to capture physiological and motion-related data of the user. In particular, optical and bioelectrical sensors may be configured to measure components of blood flow or related bio-signals in order to derive health-related attributes such as heart rate, heart-rate variability, oxygen saturation, or other cardiovascular and fitness indicators.

Further, the majority of currently known wearable devices are designed to be positioned on peripheral body locations such as the wrists, fingers, and forearms. However, these peripheral regions are especially susceptible to significant motion-induced artifacts, inconsistent and transient skin contact, and interference from sweat or environmental conditions, particularly during exercise or daily activities involving repetitive or abrupt limb movements. The underlying anatomical characteristics of these locations, such as thinner skin, prominent bones and tendons, and comparatively reduced blood perfusion, further intensify noise sensitivity and degrade signal quality for optical and electrical measurements. Therefore, these currently known wearable devices must continuously compensate for the noise and motion-induced artifacts induced in the captured signals, rendering such solutions hardware and processing-intensive. Furthermore, since the noise induced in the captured signal is greater and continuous, requiring processing to actively compensate for such noise leads to inaccurate estimation of health parameters (e.g., heart rate and variability), frequent signal dropouts, and inaccuracies in the presented health information.

Furthermore, other existing wearable devices that are designed to be positioned above the shoulders of the user, such as in-ear or ear-worn devices or neck-worn devices, offer advantages over those worn on peripheral body locations like the wrists, fingers, or forearms by being less susceptible to motion-induced artifacts and noise in the captured signal during physical activities. However, the existing wearable devices, positioned above the user's shoulders for health and fitness monitoring, also exhibit significant limitations. These wearable devices positioned on the ear (e.g., in the in-ear canal or behind-the-ear) often suffer from motion-induced artifacts caused by jaw movements during speaking, chewing, running, yawning, workout sessions, and similar activities. These jaw-induced motions deform the ear-canal tissues and introduce signal distortions, i.e., noise, into the captured signals. As a result, the monitored health and fitness data may exhibit higher errors during such activities or require substantial processing to compensate for the noise. These existing devices are unable to differentiate between the various types of movements that generate such artifacts and therefore fail to provide tailored mechanisms to adequately compensate for noise based on its underlying cause, thereby rendering them inadequate for reliable physiological monitoring during real-world activities/day-to-day operations.

Additionally, the existing wearable devices positioned on the ear suffer significantly from noise induced in the captured signal due to sweat while monitoring the health and fitness data of the user. Further, said wearable devices, particularly ear-worn devices, also face challenges with consistent fit, occlusion discomfort, wax buildup, and pressure-induced pain during prolonged wear, limiting usability for continuous monitoring, which renders these wearable devices unfit for daily/continuous use. Additionally, neck-worn devices are more prone to artifacts from head tilting, swallowing, or clothing friction, alongside heightened vulnerability to sweat and skin slippage. Furthermore, since the neck lacks a flat surface and bony prominence for secure sensor contact, it introduces greater challenges with inconsistent skin coupling and occlusion issues.

Thus, there exists an imperative need in the art to provide a technical solution for the above-mentioned and such other technical limitations of the existing solutions.

### SUMMARY OF DISCLOSURE

This section is provided to introduce certain aspects of the present disclosure in a simplified form that are further described below in the detailed description. This summary is not intended to identify the key features or the scope of the claimed subject matter.

According to an aspect, the present disclosure relates to a temple mounted wearable device. The temple mounted wearable device includes a housing unit and a computing unit disposed within the housing unit. The housing unit includes at least an aperture positioned to expose a sensor unit to a skin overlying a superficial temporal artery (STA) at a temple region of a user. The computing includes at least the sensor unit and a processor connected to at least the sensor unit. The sensor unit is configured to detect a set of physiological data from the STA, and the processor is configured to process the set of physiological data.

In an exemplary aspect of the present disclosure, the housing unit includes a first housing member and a second housing member including the aperture.

In an exemplary aspect of the present disclosure, the second housing member is configured to conform to a shape of the temple region of the user.

In an exemplary aspect of the present disclosure, the sensor unit includes at least one optical sensor and at least one inertial measurement unit (IMU) sensor, wherein the at least one optical sensor includes at least one photoplethysmography (PPG) sensor.

In an exemplary aspect of the present disclosure, the set of physiological data includes at least a set of photoplethysmography (PPG) data and a set of inertial measurement unit (IMU) data, and wherein the set of IMU data includes a set of first IMU data associated with a head region of the user, and a set of second IMU data associated with one or more regions below the head region of the user.

In an exemplary aspect of the present disclosure, the second housing member includes at least an inner layer and an outer layer. The outer layer includes at least a set of protrusions and a sticking component configured to secure the temple mounted wearable device to the temple region of the user.

In an exemplary aspect of the present disclosure, the set of protrusions and the sticking component defines one or more vents.

In an exemplary aspect of the present disclosure, the housing unit includes a housing assembly member that uses at least an interlocking technique and an adhesive technique.

In an exemplary aspect of the present disclosure, the first housing member and the second housing member define a housing cavity for housing the computing unit.

In an exemplary aspect of the present disclosure, the temple mounted wearable device further includes an insulation layer coupled to at least the computing unit, wherein the insulation layer is configured to insulate the user from a set of electromagnetic (EM) waves associated with the temple mounted wearable device.

In an exemplary aspect of the present disclosure, to process the set of physiological data, the processor is configured to receive from the sensor unit the set of physiological data associated with the user. Further, the processor is configured to determine a motion correction status associated with the set of PPG data based on the set of IMU data, wherein the motion correction status is one of a positive motion correction status and a negative motion correction status. Further, the processor is configured to generate a set of enhanced PPG data based on the set of IMU data in an event, and the positive motion correction status is determined. Further, the processor is configured to extract one or more blood flow indicators associated with the user based on at least one of the set of PPG data and the set of enhanced PPG data, wherein extracting the one or more blood flow indicators is further based on the motion correction status. The processor is further configured to generate a set of health metrics associated with the user based on the one or more blood flow indicators. The processor is further configured to monitor the set of health attributes associated with the user based on the set of health metrics. Thereafter, the processor is configured to determine a health state associated with the user based on the set of health attributes.

In an exemplary aspect of the present disclosure, the set of health attributes is associated with at least one of one or more heart rate attributes, one or more sleep attributes, one or more respiratory attributes, one or more brain attributes and one or more blood flow attributes.

According to another aspect, the present disclosure relates to a method for monitoring the set of health attributes associated with the user. The method includes receiving the set of physiological data associated with the user, wherein the set of physiological data includes at least one of the set of photoplethysmography (PPG) data and the set of inertial measurement unit (IMU) data. Further, the method includes determining the motion correction status associated with the set of PPG data based on the set of IMU data, wherein the motion correction status indicates one of the positive motion correction status and the negative motion correction status. Further, the method includes generating the set of enhanced PPG data based on the set of IMU data in an event the positive motion correction status is determined. Furthermore, the method includes extracting the one or more blood flow indicators associated with the user based on at least one of the set of PPG data and the set of enhanced PPG data. The method further includes generating the set of health metrics associated with the user based on the one or more blood flow indicators. Thereafter, the method includes monitoring the set of health attributes associated with the user based on the set of health metrics for determining the health state associated with the user.

In an exemplary aspect of the present disclosure, the set of physiological data is detected from the superficial temporal artery (STA) associated with the temple region of the user.

In an exemplary aspect of the present disclosure, the method further includes determining a temporal alignment status associated with the set of IMU data and the set of PPG data, wherein the temporal alignment status is one of a positive temporal alignment status and a negative temporal alignment status. Further, the method includes determining in the set of physiological data, a set of IMU weights associated with the set of IMU data in an event, and determining the positive temporal alignment status. Thereafter, the method includes determining the motion correction status associated with the set of PPG data based on the set of IMU weights.

In an exemplary aspect of the present disclosure, the positive motion correction status associated with the set of PPG data is determined in an event one or more weight values in the set of IMU weights is one of equal to a first predefined threshold value and above the first predefined threshold value.

In an exemplary aspect of the present disclosure, the negative motion correction status associated with the set of PPG data is determined in an event one or more weight values in the set of IMU weights is one of below the first predefined threshold value.

In an exemplary aspect of the present disclosure, the positive temporal alignment status associated with the set of IMU data and the set of PPG data is determined in an event a set of IMU data time-period values associated with the set of IMU data is equal to a set of PPG data time-period values associated with the set of PPG data, and the negative temporal alignment status associated with the set of IMU data and the set of PPG data is determined in an event the set of IMU data time-period values associated with the set of IMU data differs from the set of PPG data time-period values associated with the set of PPG data.

In an exemplary aspect of the present disclosure, the set of IMU data includes one or more of a set of first IMU data associated with a head region of the user, and a set of second IMU data associated with one or more regions below the head region of the user.

In an exemplary aspect of the present disclosure, the method further includes determining in the set of IMU data, at least one of a set of first IMU weight associated with the set of first IMU data and a set of second IMU weight associated with the set of second IMU data in an event the positive motion correction status is determined. Further, the method includes identifying from the set of IMU data, a set of target IMU data based on the set of first IMU weight and the set of second IMU weight and a second predefined threshold value. Thereafter, the method includes generating the set of enhanced PPG data based on the set of target IMU data.

In an exemplary aspect of the present disclosure, the set of first IMU data is identified as the set of target IMU data in an event a value of the set of first IMU weight in the set of IMU data is one of equal to the second predefined threshold value and above the second predefined threshold value, and the set of second IMU data is identified as the set of target IMU data in an event a value of the set of second IMU weight in the set of IMU data is one of equal to the second predefined threshold value and above the second predefined threshold value.

In an exemplary aspect of the present disclosure, the method further includes filtering the set of first IMU data from the set of IMU data, in an event the identified set of target IMU data includes the set of first IMU data. Further, the method includes determining a set of first IMU attributes associated with the set of first IMU data, wherein the set of first IMU attributes includes at least one of a set of first motion attributes and a set of first orientation attributes. Further, the method includes identifying a set of first user activity types associated with the set of first IMU data based on the set of first IMU attributes, wherein the set of first user activity types includes at least one of a facial muscle activity and a non-facial muscle activity. Further, the method includes filtering from the set of first IMU data, a set of facial muscle activity IMU data and a set of non-facial muscle activity IMU data based on the set of first user activity types. Thereafter, the method includes generating the set of enhanced PPG data based on at least one of the set of facial muscle activity IMU data and the set of non-facial muscle activity IMU data.

In an exemplary aspect of the present disclosure, the set of facial muscle activity IMU data is associated with one or more facial muscles of the user, and wherein the set of non-facial muscle activity IMU data is associated with one or more body part movement associated with the head region of the user.

In an exemplary aspect of the present disclosure, the method further includes fetching a first set of error correction values from a set of predefined error correction values based on the set of facial muscle activity IMU data in an event the identified set of first user activity types includes the facial muscle activity. Thereafter, the method includes generating the set of enhanced PPG data based on the first set of error correction values.

In an exemplary aspect of the present disclosure, the method further includes determining a set of non-facial IMU attributes associated with the non-facial muscle activity IMU data in an event the identified set of first user activity types includes the non-facial muscle activity. Further, the method includes determining in the set of first IMU data, a set of non-facial IMU weights associated with the set of non-facial muscle activity IMU attributes. Further, the method includes generating a set of second error correction values associated the non-facial muscle activity based on the set of non-facial IMU weights. Thereafter, the method includes enhancing the set of PPG data based on the set of second error correction values.

In an exemplary aspect of the present disclosure, the method further includes filtering the set of second IMU data from the set of IMU data in an event the identified set of target IMU data includes the set of second IMU data. Further, the method includes determining a set of second IMU attributes associated with the set of second IMU data, wherein the set of second IMU attributes includes a set of second motion attributes associated with the set of second IMU data and a set of second orientation attributes associated with the set of second IMU data. The method further includes generating a set of third error correction values associated with the set of second IMU data based on the set of second IMU attributes. Thereafter, the method includes enhancing the set of PPG data based on the set of third error correction values.

In an exemplary aspect of the present disclosure, for extracting the one or more blood flow indicators, the method further include identifying at least one of one or more temporal features associated with the user based on at least one of the set of PPG data and the set of enhanced PPG data, wherein the one or more temporal features are identified based on the set of PPG data in an event the negative motion correction status is determined and the one or more temporal features are identified based on the set of enhanced PPG data in an event the positive motion correction status is determined. Further, extracting the one or more blood flow indicators also includes determining a set of first indicators associated with the user based on the one or more temporal features. Further, extracting the one or more blood flow indicators also includes identifying one or more amplitude features associated with the user based on at least one of the set of PPG data and the set of enhanced PPG data, wherein the one or more amplitude features are identified based on the set of PPG data in an event the negative motion correction status is determined and the one or more amplitude features are identified based on the set of enhanced PPG data in an event the positive motion correction status is determined. Thereafter, extracting the one or more blood flow indicators includes determining a set of second indicators associated with the user based on the one or more amplitude features.

In an exemplary aspect of the present disclosure, the method further includes generating the set of health metrics based on one or more of the set of first indicators and the set of second indicators, wherein the set of health metrics includes at least one of one or more heart rate metrics, one or more sleep metrics and one or more blood flow metrics.

In an exemplary aspect of the present disclosure, the one or more temporal features include at least one of a beat length feature, a data periodicity feature, a dicrotic notch feature, a set of phase shift features, a set of derivative features, and a set of transient variation features.

In an exemplary aspect of the present disclosure, the set of first indicators includes at least one of one or more cerebral blood flow (CBF) indicators and one or more brain activity indicators.

In an exemplary aspect of the present disclosure, the one or more amplitude features include at least one of a set of data intensity features, a set of peak-to-peak value features, a set of baseline shift features and a set of pulsatility index features.

In an exemplary aspect of the present disclosure, the set of second indicators includes at least one of a set of cerebral blood volume (CBV) indicators, a set of brain-wave indicators and a set of physiological response features.

In an exemplary aspect of the present disclosure, the set of health attributes is associated with at least one of one or more heart rate attributes associated with the user, one or more sleep attributes associated with the user and one or more blood flow attributes associated with the user.

In an exemplary aspect of the present disclosure, the method further includes receiving in real-time from the temple region of the user, a set of target PPG data, wherein the set of target PPG data includes at least one of the set of PPG data and the set of enhanced PPG data. Further, the method includes extracting in real-time, the one or more blood flow indicators associated with the user based on at least the set of real-time target PPG data. Further, the method includes determining a set of real-time health metric values associated with the user based on one or more real-time blood flow indicators. Thereafter, the method includes monitoring the set of health attributes associated with the user based on comparing the set of real-time health metric values and one or more values associated with the set of health metrics to determine the health state of the user.

According to yet another aspect, the present disclosure relates to a system for monitoring the set of health attributes of the user. The system includes the temple mounted wearable device configured to be positioned at a temple region of the user. Said temple mounted wearable device includes the sensor unit and the processor. The sensor unit includes the at least one set of optical sensor and the at least one inertial measurement unit (IMU) sensor. The at least one optical sensor is configured to detect the set of photoplethysmography (PPG) data from the superficial temporal artery (STA) at the temple region, and the at least one IMU sensor is configured to detect the set of inertial measurement unit (IMU) data associated with the user. The processor is configured to perform the real-time motion artifact correction on the set of PPG data using the set of IMU data, and to derive the one or more blood flow indicators from at least one of the set of PPG data and the set of enhanced PPG data. The system includes a user device communicatively coupled to the temple mounted wearable device and is configured to receive the one or more blood flow indicators. The system further includes a server communicatively coupled to the user device and is configured to generate the set of health metrics associated with the user based on the one or more blood flow indicators, and to determine the health state of the user based on the set of health metrics.

According to yet another aspect, the present disclosure relates to a non-transitory computer readable storage medium storing instructions for monitoring the set of health attributes associated with the user, the instructions including executable code which when executed by the processor, causes the processor to perform operations including receiving the set of physiological data associated with the user, wherein the set of physiological data includes at least one of the set of photoplethysmography (PPG) data and the set of inertial measurement unit (IMU) data. Further, the operations include determining the motion correction status associated with the set of PPG data based on the set of IMU data, wherein the motion correction status indicates one of the positive motion correction status and the negative motion correction status. Further, the operations include generating the set of enhanced PPG data based on the set of IMU data in an event the positive motion correction status is determined. Furthermore, the operations include extracting the one or more blood flow indicators associated with the user based on at least one of the set of PPG data and the set of enhanced PPG data. The operations further include generating the set of health metrics associated with the user based on the one or more blood flow indicators. Thereafter, the operations include monitoring the set of health attributes associated with the user based on the set of health metrics for determining the health state associated with the user.

### BRIEF DESCRIPTION OF DRAWINGS

The accompanying drawings, which are incorporated herein, constitute a part of this disclosure. Components in the drawings are not necessarily to scale, emphasis instead being placed upon clearly illustrating the principles of the present disclosure. Some drawings may indicate the components using block diagrams and may not represent the internal circuitry of each component. It will be appreciated by those skilled in the art that disclosure of such drawings includes disclosure of electrical components or circuitry commonly used to implement such components. Although exemplary connections between sub-components have been shown in the accompanying drawings, it will be appreciated by those skilled in the art that other connections may also be possible, without departing from the scope of the disclosure. All sub-components within a component may be connected to each other, unless otherwise indicated.

FIG. 1A illustrates an exemplary environment in which a temple mounted wearable device operates, in accordance with the embodiments of the present disclosure;

FIG. 1B illustrates an exemplary block diagram of the temple mounted wearable device, in accordance with the exemplary embodiments of the present disclosure;

FIG. 2 illustrates an exemplary perspective view depicting exemplary placement of the temple mounted wearable device on a user, in accordance with the exemplary embodiments of the present disclosure;

FIG. 3A illustrates an exemplary shape of the temple mounted wearable device, in accordance with the exemplary embodiments of the present disclosure;

FIG. 3B illustrates another exemplary shape of the temple mounted wearable device, in accordance with the exemplary embodiments of the present disclosure;

FIG. 4 illustrates an exemplary cross-section view of the temple mounted wearable device, in accordance with the exemplary embodiments of the present disclosure;

FIG. 5 illustrates another exemplary cross-sectional view of the temple mounted wearable device, in accordance with the exemplary embodiments of the present disclosure;

FIG. 6 illustrates an exemplary exploded isometric view of the temple mounted wearable device, in accordance with the exemplary embodiments of the present disclosure;

FIG. 7 illustrates an exemplary flow diagram of a method for monitoring a set of health attributes associated with the user, in accordance with the exemplary embodiments of the present disclosure; and

FIG. 8 illustrates an exemplary processing pipeline implemented by the temple mounted wearable device for monitoring the set of health attributes associated with the user, in accordance with the exemplary embodiments of the present disclosure.

The foregoing shall be more apparent from the following more detailed description of the disclosure.

### DETAILED DESCRIPTION

In the following description, for the purposes of explanation, various specific details are set forth in order to provide a thorough understanding of embodiments of the present disclosure. It will be apparent, however, that embodiments of the present disclosure may be practiced without these specific details. Several features described hereafter may each be used independently of one another or with any combination of other features. An individual feature may not address any of the problems discussed above or might address only some of the problems discussed above.

The ensuing description provides exemplary embodiments only, and is not intended to limit the scope, applicability, or configuration of the disclosure. Rather, the ensuing description of the exemplary embodiments will provide those skilled in the art with an enabling description for implementing an exemplary embodiment. It should be understood that various changes may be made in the function and arrangement of elements without departing from the spirit and scope of the disclosure as set forth.

Specific details are given in the following description to provide a thorough understanding of the embodiments. However, it will be understood by one of ordinary skills in the art that the embodiments may be practiced without these specific details. For example, circuits, systems, processes, and other components may be shown as components in block diagram form in order not to obscure the embodiments in unnecessary detail.

Also, it is noted that individual embodiments may be described as a process which is depicted as a flowchart, a flow diagram, a data flow diagram, a structure diagram, or a block diagram. Although a flowchart may describe the operations as a sequential process, many of the operations may be performed in parallel or concurrently. In addition, the order of the operations may be re-arranged. A process is terminated when its operations are completed but could have additional steps not included in a figure.

The word “exemplary” and/or “demonstrative” is used herein to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, such terms are intended to be inclusive in a manner similar to the term “comprising” as an open transition word without precluding any additional or other elements.

As used herein, an “apparatus” or an “electronic apparatus” or a “wearable apparatus” may refer to a set of devices used for the implementation of the technical solution provided by the present disclosure. Said apparatus may include one or more wearable devices that may either individually or collectively perform one or more functions for implementing the technical solutions of the present disclosure. The term “wearable apparatus” may refer to a device/computer that may be worn by a user 101 of the wearable apparatus.

Further, as used herein, the terms “device,” “wearable device,” “temple-mounted device,” “temple-mounted wearable device,” or “electronic device” refer to a head-worn wearable unit including one or more components or modules that enable the implementation of the technical solutions of the present disclosure. In exemplary embodiments, the wearable device is configured in a compact form factor dimensioned to conform ergonomically to the anatomical contour of a temple region 142 of the human subject. In such exemplary implementations, the wearable device is positioned so as to overlie an artery, such as a superficial temporal artery, and adjacent vascular structures, and is adhered to the skin of the user at the temple region 142 using a latching or sticking mechanism, such as a biocompatible adhesive layer, to ensure stable and consistent skin-device contact.

As used herein, a “module” or a “processing unit” includes one or more processors, wherein processor refers to any logic circuitry for processing instructions. A processor may be a general-purpose processor, a special purpose processor, a conventional processor, a digital signal processor, a plurality of microprocessors, one or more microprocessors in association with a DSP core, a controller, a microcontroller, Application Specific Integrated Circuits, Field Programmable Gate Array circuits, any other type of integrated circuits, etc. The processor may perform signal coding, data processing, input/output processing, and/or any other functionality that enables the working of the system according to the present disclosure. More specifically, the processor or processing unit is a hardware processor.

As used herein, “a user device” may be any electrical, electronic, and/or computing device or equipment capable of implementing the features of the present disclosure. The user equipment/device may include, but is not limited to, a mobile phone, smart phone, laptop, a general-purpose computer, desktop, personal digital assistant, tablet computer, the wearable device or any other computing device that is capable of implementing the features of the present disclosure. Also, the user device may contain at least one input means configured to receive an input from at least one of a transceiver unit, a processing unit, a storage unit, a detection unit and any other such unit(s) which are required to implement the features of the present disclosure.

As used herein, a “printed circuit board” or “PCB” includes any circuit-bearing substrate that supports conductive pathways and electronic components. The PCB may be a rigid board, a flexible circuit, a rigid-flex board, a multilayer construction, a high-density interconnect (HDI) board, a metal-core board, a ceramic substrate, an additively manufactured electronic substrate, or any other substrate capable of supporting conductive traces, vias, pads, components, or interconnects. Further, as used herein, the PCB may incorporate various structural or material features, including different layer counts, dielectric materials, trace widths or geometries, via structures, stackups, surface finishes, and component-mounting configurations. Such PCB may further support analog, digital, RF, microwave, mixed-signal, power-distribution, or sensor circuitry and may employ surface-mount, through-hole, embedded, or integrated components. Further, the PCB may be fabricated using etching, deposition, printing, lamination, machining, laser processing, or any other known or future-developed manufacturing technique. More specifically, the PCB may be a hardware substrate that provides electrical interconnection and mechanical support for electronic components.

As used herein, the term “optical sensor(s)” may include any light-based sensing module that detects reflected, transmitted, or scattered light to derive physiological information. The optical sensor(s) may include, without limitation, photoplethysmography (PPG) sensors, reflectance-based or transmission-based sensors, laser or LED emitters operating at red, green, infrared, or near-infrared wavelengths, photodiodes, CMOS image sensors, multi-wavelength or multi-LED arrays, speckle-plethysmography (SPG) sensors, or any optical module configured to measure physiological parameters such as heart rate, blood oxygen saturation (SpO₂), heart rate variability (HRV), perfusion, respiration, or hydration in health and fitness monitoring systems. Further, as used herein, the optical sensor(s) may incorporate various structural, material, or operational features, including different emitter wavelengths, detector types, modulation schemes, signal-processing algorithms, integration with accelerometers or other sensing modalities, skin-tone correction techniques, sampling rates, or mounting positions. Said optical sensor(s) may further employ coherent or incoherent light sources, single-channel or multi-channel detection, contact-based or remote configurations, analog or digital processing, motion-artifact rejection, or multi-sensor fusion with modalities such as impedance plethysmography (IPG) or electrocardiography (ECG). Further, such optical sensor(s) may be fabricated using etching, deposition, printing, lamination, integration into wearable devices such as rings, wristbands, or earpieces, laser processing, or any other known or future-developed manufacturing method. More specifically, the optical sensor(s) may be configured as a hardware sensing module that uses optical energy to acquire physiological data from a particular user.

As used herein, the “user device” and/or “wearable device” and/or “module” may include at least “storage unit” or “memory unit”, wherein “storage unit” or “memory unit” refers to a machine or computer-readable medium including any mechanism for storing information in a form readable by a computer or similar machine. For example, a computer-readable medium includes read-only memory (“ROM”), random access memory (“RAM”), magnetic disk storage media, optical storage media, flash memory devices or other types of machine-accessible storage media. The storage unit stores at least the data that may be required by one or more units of the system to perform their respective functions.

As used herein, the expression “and/or” includes any single item from items or a combination of items associated with the items. For example, a group of A, B and/or C includes only A, only B, only C, a combination of A and B, a combination of B and C, a combination of A and C or a combination of A, B and C.

As used herein, the expression “a set of” shall be interpreted as a collection of datasets or elements. The set may include a finite set with one element or more than one element. In an example, a set of elements may be made by an array of elements. For example, “a set of X” includes {X}, {XX} and/or {X1, X2, X3}. Further, “the set of X” may also include an element Y that may or may not be related to X unless repugnant to the context thereof.

As used herein, the expression “one or more of” includes any single item in the list or a combination of items in the list. For example, one or more of A, B and C includes only A, only B, only C, a combination of A and B, a combination of B and C, a combination of A and C, a combination of A, B and C, a combination of multiple A and multiple B, a combination of multiple A, a single B and a single C, a combination of single A, multiple B and multiple C and any other such like combinations.

As used herein, the expression “at least one of” shall be interpreted as an inclusive term that includes at least one of the succeeding elements, and shall also include multiple such elements in different combinations. For example, the term “an exemplary parameter including at least one of A, B and C” may imply that the exemplary parameter may include only {A} or only {B} or only {C}, or {A, B, C} collectively, and may also include various combinations of A, B, and C (with or without any other element such as D), such as {A, B}, {B, C}, {C, A}, {C, D}, {D, A} and any other such like combinations. Further, the expression shall also be construed to include multiple instances of such elements, for example {A, A}, {A. A, D}, {A, B, C, A, B, C}, {A, B, C, A, B, C, D}, etc.

The present subject matter is further described with reference to the accompanying figures. Wherever possible, the same reference numerals are used in the figures and the following description to refer to the same or similar parts. It should be noted that the description and figures merely illustrate principles of the present subject matter. It is thus understood that various arrangements may be devised that, although not explicitly described or shown herein, encompass the principles of the present subject matter. Moreover, all statements herein reciting principles, aspects, and examples of the present subject matter, as well as specific examples thereof, are intended to encompass equivalents thereof.

As discussed in the background section, current known solutions exhibit several shortcomings. The present disclosure addresses these and other existing problems in the field by providing a temple mounted wearable device 100 and a method for monitoring health attributes associated with the user 101. The novel and inventive temple mounted wearable device 100, as disclosed by the present disclosure, is configured for placement on the temple region 142 of the user 101, a location that offers improved signal stability compared to peripheral body regions. The temple mounted wearable device 100 includes an adaptive housing with the set of protrusions 110 that, in combination with a sticking component, securely attach the temple mounted wearable device 100 to the temple region 142 of the user 101, while maintaining stable sensor contact and allowing venting to reduce sweat-induced noise. The present disclosure further distinguishes between different sources and magnitudes of noise in the captured signals, including subtle head-region movements, general head motion, and vigorous limb activity. The present disclosure, based on the identified source of noise, filters the monitored data and, when noise exceeds a predefined threshold, selectively reduces processing requirements. The present disclosure stabilizes sensor placement and intelligently detects and manages motion-induced artifacts, enabling reliable, real-time derivation of key health attributes such as heart rate, sleep patterns, respiration, and cerebral blood-flow metrics, while relying on simpler hardware and reduced processing complexity compared to existing systems.

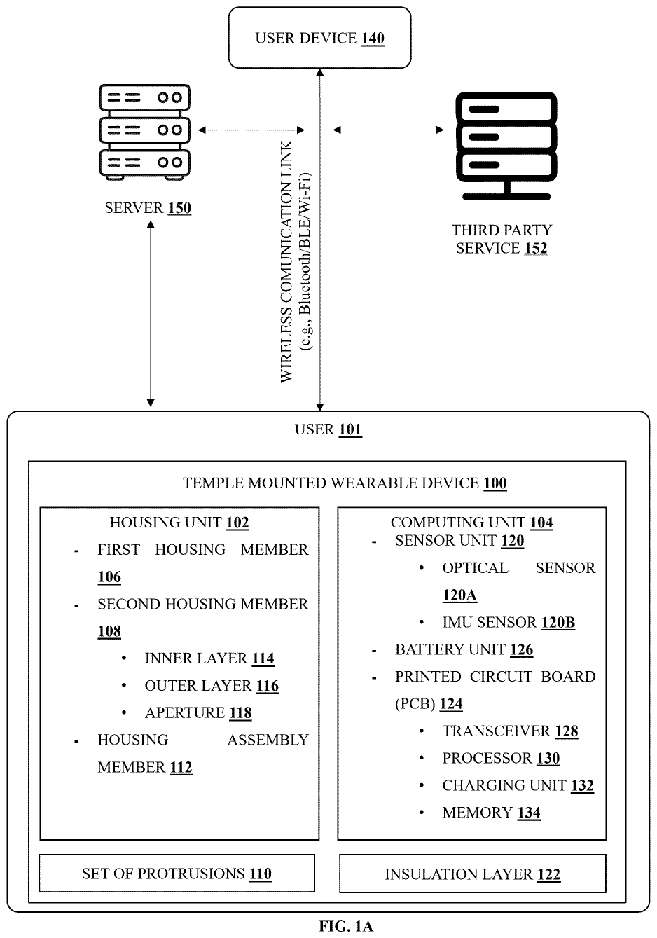

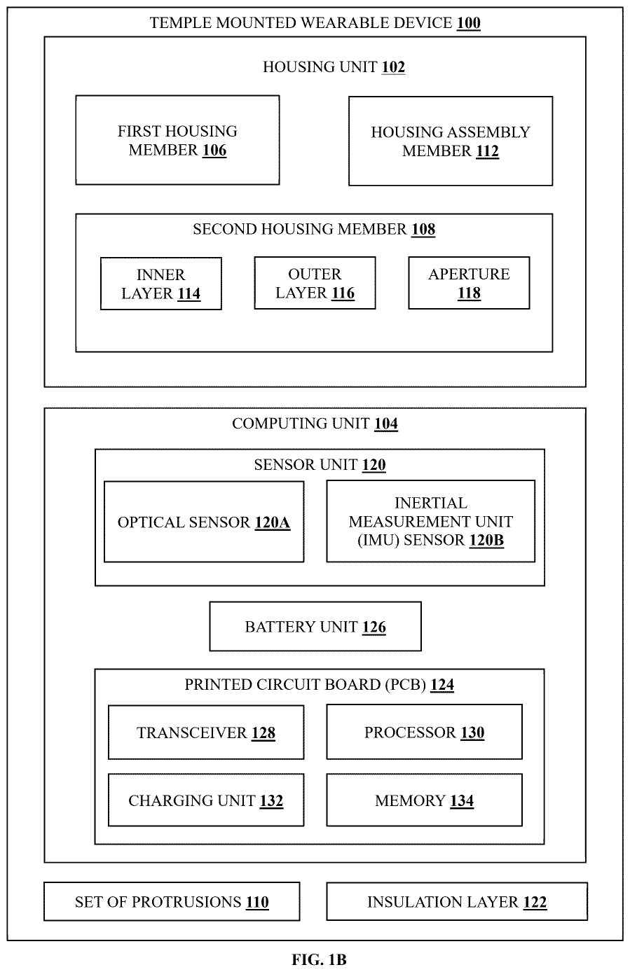

FIG. 1A illustrates an exemplary environment in which the temple mounted wearable device 100 operates, in accordance with the embodiments of the present disclosure. As shown, the temple mounted wearable device 100 includes a housing unit 102 including at least a first housing member 106 and a second housing member 108 and a housing assembly member 112 configured to join the first housing member 106 and the second housing member 108. Further, the second housing member 108 includes an inner layer 114, an outer layer 116, and an aperture 118. The temple mounted wearable device 100 further includes a computing unit 104 disposed within the housing unit 102, the computing unit 104 including a sensor unit 120, a printed circuit board (PCB) 124 and a battery unit 126. The sensor unit 120 includes at least one optical sensor 120A and at least one inertial measurement unit (IMU) sensor 120B configured to monitor a set of physiological data associated with the user 101. In an implementation, the at least one optical sensor 120A includes at least one photoplethysmography (PPG) sensor. The PCB 124 includes at least a transceiver 128, a processor 130, a charging unit 132 and a memory 134. The temple mounted wearable device 100 additionally includes the set of protrusions 110 and an insulation layer 122. Further, said set of protrusions 110, in combination with the sticking component, defines one or more vents. Further, the one or more vents are configured to reduce sweat accumulation and facilitate thermal regulation at the interface between the temple mounted wearable device 100 and the skin of the user 101.

As depicted in FIG. 1A, the temple mounted wearable device 100 is configured to communicate with a user device 140, such as a smartphone or tablet, via a wireless communication link that may include at least one of Bluetooth, Bluetooth Low Energy (BLE), Wi-Fi, or any other wireless communication mechanism apparent to a person skilled in the art. The user device 140 may be configured to communicate with a server 150, which may be implemented as a cloud-based or remote computing platform. The server 150 may communicate with one or more third-party services 152 that provide additional processing, analytics, or application-specific functionalities associated with the monitoring of a set of health attributes of the user 101. The bidirectional communication links as illustrated in FIG. 1A represents the exchange of data between the temple mounted wearable device 100, the user device 140, the server 150, and the third-party services 152, thereby depicting the operational ecosystem in which the temple mounted wearable device 100 functions to implement the features of the present disclosure.

In certain embodiments, the server 150 may be further configured to communicate with the third-party services 152 that may operate as external platforms or systems providing supplementary processing or application-specific functionalities. In such embodiments, the third-party services 152 may receive the set of physiological data and an anonymized analytical outputs (such as user identifier, operation tag identifier or similar anonymized analytical outputs) from the server 150 to perform additional operations that extend the capabilities of the temple mounted wearable device 100. For example, the third-party services 152 may include cloud-based analytics engines configured to execute advanced signal-processing algorithms, machine-learning models, or long-term trend analyses that exceed the computational capacity of the temple mounted wearable device 100 or the user device 140. In other embodiments, the third-party services 152 may include wellness or health-monitoring platforms that generate personalized insights, behavioral recommendations, or user-specific reports based on monitored health attributes over a predefined period of time, such as the last 7 days, last 3 months, or between 8 PM and 6 AM. In further embodiments, the third-party services 152 may include secure data-storage systems that archive historical physiological data for retrospective analysis, compliance, or longitudinal health tracking. Additionally, the third-party services 152 may encompass telehealth or clinical-integration platforms that enable optional communication with healthcare providers, subject to authorization by the user 101. These embodiments illustrate that the third-party services 152 may provide any form of auxiliary processing, storage, or application-level functionality that enhances or extends the monitoring and interpretation of the set of health attributes associated with the user 101, without limiting the scope of the present disclosure.

Referring to FIG. 1B, an exemplary block diagram of the temple mounted wearable device 100, in accordance with the exemplary embodiments of the present disclosure, is illustrated. The temple mounted wearable device 100 includes the housing unit 102, the computing unit 104, the set of protrusions 110 and the insulation layer 122. Further, as depicted in FIG. 1B the housing unit 102 includes the first housing member 106, the second housing member 108 and the housing assembly member 112. Also, the second housing member 108 includes the inner layer 114, the outer layer 116 and the aperture 118. Further, the computing unit 104 includes the sensor unit 120, the printed circuit board (PCB) 124 and the battery unit 126. Further, in a preferred implementation, the sensor unit 120 may include the at least one optical sensor 120A and the at least one inertial measurement unit (IMU) sensor 120B. Furthermore, the PCB 124 includes at least the transceiver 128, the processor 130, the charging unit 132 and the memory 134. All the units in the temple mounted wearable device 100 are communicatively coupled to each other in a manner as obvious to a person skilled in the art for implementing features of the present disclosure. Also, in FIG. 1B only a few units are shown, however, the temple mounted wearable device 100 may include multiple such units, as may be required to implement the features of the present disclosure. Further, in an embodiment, the temple mounted wearable device 100 may be connected to and/or in communication with a user device (may also be referred herein as a user equipment or a UE or the user device 140) to implement the features of the present disclosure. Furthermore, in another embodiment, the temple mounted wearable device 100 may be connected to and/or in communication with a server (the server 150) to implement the features of the present disclosure.

In an exemplary implementation, the present disclosure includes a system for monitoring the set of health attributes of the user 101. The system includes the temple mounted wearable device 100 configured to be positioned at the temple region 142 of the user 101, said temple mounted wearable device 100 including the sensor unit 120 that includes the at least one optical sensor 120A and the at least one inertial measurement unit (IMU) sensor 120B, wherein the at least one optical sensor 120A is configured to detect a set of photoplethysmography (PPG) data from a superficial temporal artery (STA) at the temple region 142 and the at least one inertial measurement unit (IMU) sensor 120B is configured to detect a set of inertial measurement unit (IMU) data associated with the user 101. Further, the temple mounted wearable device 100 includes the processor 130 configured to perform a real-time motion artifact correction on the set of PPG data using the set of IMU data, and to derive one or more blood flow indicators from the set of PPG data. The system further includes the user device 140 communicatively coupled to the temple mounted wearable device 100, and is configured to receive the one or more blood flow indicators. The system further includes the server 150 communicatively coupled to the user device 140 and configured to generate a set of health metrics associated with the user based on the one or more blood flow indicators, and to determine a health state of the user based on the set of health metrics.

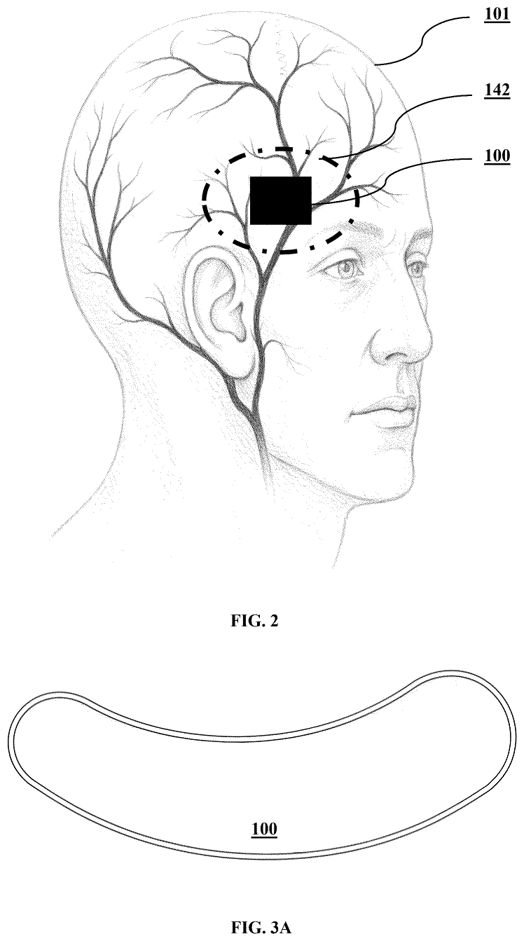

Further, referring to FIG. 2, which illustrates an exemplary perspective view depicting exemplary placement of the temple mounted wearable device 100 on the user 101, in accordance with the exemplary embodiments of the present disclosure. As shown in FIG. 2, the temple mounted wearable device 100 for monitoring the set of health attributes associated with the user 101 may be placed on the temple region 142 of the user 101. In a preferred implementation of the present disclosure, the temple mounted wearable device 100 is placed on the superficial temporal artery (STA) associated with the temple region 142 of the user 101. Further, in an exemplary implementation, the sensor unit 120 in the temple mounted wearable device 100 is configured to detect the set of physiological data from the superficial temporal artery (STA) associated with the temple region 142.

It may be appreciated by a person skilled in the art that the term “temple region 142” as indicated in FIG. 2 is merely exemplary and should not be interpreted to limit the scope of the present disclosure. Further, in an alternate embodiment, the temple region 142 includes a frontal temple region of the user, including an anatomical area generally located lateral to the forehead and superior to the zygomatic arch of the user 101. In an alternate embodiment, the temple region 142 may further include any physiologically comparable or adjacent region around the frontal temple region that would be reasonably appreciated by a person skilled in the art as suitable for placement of the temple mounted wearable device 100.

In an alternate embodiment, it may be appreciated by a person skilled in the art that the temple region 142 may include anatomical locations along or proximate to Pitanguy's line, which extends from the tragus towards a lateral eyebrow and is commonly used as a clinical reference for identifying a course of the STA and associated neurovascular structures. Further, it is to be noted that the placement of the temple mounted wearable device 100 on and around the Pitanguy's line is intended solely as an exemplary anatomical reference and does not limit the scope of the temple region 142. Accordingly, the temple region 142 encompasses regions overlying or adjacent to the STA, as well as any similar anatomical locations that provide adequate access to vascular, neurological, or soft-tissue structures from which the sensor unit 120 may detect the set of physiological data.

Furthermore, in certain embodiments of the present disclosure, the temple mounted wearable device 100 may be positioned at the anatomical locations that provide access to an internal carotid artery (ICA) of the user 101 for detecting the set of physiological data. As may be appreciated by a person skilled in the art, the ICA supplies intracranial structures and carries hemodynamic information reflective of cerebral circulation. Accordingly, placement of the temple mounted wearable device 100 at regions overlying or proximate to the ICA enables the sensor unit 120 to detect the set of physiological data influenced by ICA-related blood flow.

In an alternate embodiment, the temple mounted wearable device 100 may be positioned at the anatomical locations associated with an external carotid artery (ECA) of the user 101 for detecting the set of physiological data. It may be appreciated that the term ECA, as used herein, is intended to include any segment, branch, or hemodynamic region related to the temporal region 142 that provides suitable access for detecting the set of physiological data from the ECA of the user 101.

It is to be noted that references to the term “STA” are intended to broadly encompass vascular structures supplying blood-flow to the temple region 142. As appreciated by a person skilled in the art, the temple region 142 may include body regions associated with the head of the user 101 that receive contributions from the ECA, which primarily supplies the face and scalp, and/or hemodynamic influences associated with the ICA, which supplies intracranial structures, including the brain of the user 101. Accordingly, in certain embodiments, the set of physiological data detected from the STA may reflect composite blood-flow characteristics influenced by both ECA-derived perfusion and ICA-related cerebral circulation. This understanding is intended to be exemplary and non-limiting, and the term STA within the present disclosure includes any segment, branch, or hemodynamically related vascular region that provides access to blood-flow information influenced by either or both of the ECA and the ICA.

It will be appreciated by a person skilled in the art that the placement of the temple mounted wearable device 100 on the temple region 142 of the user 101 for monitoring the set of health attributes is merely exemplary and is not intended to limit the scope of the present disclosure. Further, it may also be appreciated by a person skilled in the art that the shape of the temple mounted wearable device 100 may be adapted for positioning on or within any suitable region on the body of the user 101 that would be apparent to a person skilled in the art for implementing the solutions described herein.

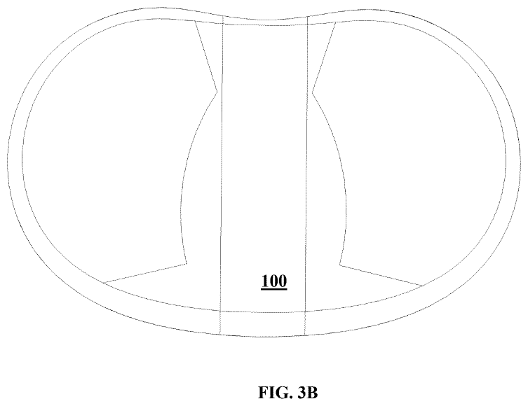

Further, referring to FIG. 3A, which illustrates an exemplary shape of the temple mounted wearable device 100, in accordance with the exemplary embodiments of the present disclosure. Furthermore, referring to FIG. 3B, which illustrates another exemplary shape of the temple mounted wearable device 100, in accordance with the exemplary embodiments of the present disclosure. In a preferred embodiment of the present disclosure, the temple mounted wearable device 100 may be a bean-shaped temple mounted wearable device 100. However, it will be appreciated by a person skilled in the art that the shape of the temple mounted wearable device 100, as illustrated in the FIG. 3A and FIG. 3B is merely exemplary and is not intended to limit the scope of the present disclosure. The temple mounted wearable device 100 may be configured in any suitable shape (such as square, round, etc.) or form factor (such as a patch or similar form factors) that would be apparent to a person skilled in the art for implementing the solution of the present disclosure as disclosed herein.

Also, for ease of understanding, FIG. 1A, FIG. 1B, FIG. 2, FIG. 3A and FIG. 3B are explained in conjunction with each other in the foregoing description for the explanation of the technical solution as disclosed by the present disclosure.

Further, the set of health attributes may be associated with at least one of one or more heart rate attributes, one or more sleep attributes, one or more respiratory attributes, one or more brain attributes and one or more blood flow attributes.

It is to be noted that the term “one or more heart rate attributes” refers to a set of data indicating one or more features/characteristics associated with the functioning of a heart of the user 101. Further, the one or more heart rate attributes may include, but is not limited to, heartbeat attribute, resting heart rate attribute, heart rate variability (HRV) attribute, maximum heart rate attribute, etc.

Further, it is to be noted that the term “one or more sleep attributes” refers to a set of data associated with sleep pattern and/or sleep quality of the user 101, such as, but is not limited to, sleep duration attribute, sleep continuity attribute, sleep timing attribute, etc.

Further, it is to be noted that the term “one or more respiratory attributes” refers to a set of data indicating one or more features/characteristics/conditions associated with a breathing pattern of the user 101, such as, but is not limited to, respiratory rate attribute, breathing frequency attribute, apnea attribute, etc.

Further, it is to be noted that the term “one or more brain attributes” refers to a set of data indicating one or more features/characteristics associated with neural and cognitive activity of the user 101, such as, but is not limited to, brainwave attributes, mental focus attributes, cognitive load attributes, stress level attributes, etc.

Furthermore, it is to be noted that the term “one or more blood flow attributes” refer to a set of data indicating one or more features/characteristics associated with blood movement in the body/brain of the user 101 such as, but is not limited to, blood flow velocity attribute, Blood Volume Pulse (BVP) attribute, blood pressure attribute, etc.

It is to be noted that the abovementioned health attributes are only exemplary in nature and in no manner intended to limit the scope of the present disclosure. The set of health attributes may include any other attribute(s) as may be appreciated by the person skilled in the art to implement the present solution.

Further, in an exemplary embodiment of the present disclosure, the housing unit 102 may refer to a physical protective shell/casing that may contain and protect all the units, members and/or modules of the temple mounted wearable device 100 that are encapsulated within the housing unit 102 of the temple mounted wearable device 100. Further, the housing unit 102 may include the first housing member 106 and the second housing member 108. Further, in an exemplary embodiment of the present disclosure, the first housing member 106 may refer to an outermost component/part of the housing unit 102, which, in combination with the second housing member 108, encapsulates/houses the computing unit 104. Further, it may be appreciated by the person skilled in the art that, in certain embodiments, the first housing member 106 and the second housing member 108 may be integrally formed as portions of a single, continuous housing structure and need not be physically separate or disjoint from one another, to implement the solution of the present disclosure.

In another embodiment, the housing unit 102 may be constructed from a material or combination of materials configured to conform to, or adapt to, the anatomical curvature/contours of the temple region 142 of the user 101. Such materials may include flexible polymers, viscoelastic materials, memory-foam-based composites, thermoplastic elastomers (TPE), thermoplastic polyurethane (TPU), silicone-based materials, flexible composite materials, metallic alloys, carbon-fiber-reinforced materials, shape-adaptive elastomers, shape-shifting materials or any combination thereof that enable the housing unit 102 to achieve a secure and comfortable fit during prolonged/continuous use of the temple mounted wearable device 100 by the user 101. In another embodiment, the housing unit 102 may be constructed from a combination of materials to enable a predetermined degree of elasticity or flexibility that enables the housing unit 102 to exert a gentle, evenly distributed pressure against the temple region 142 in order to improve stability of the temple mounted wearable device 100 during motion while minimizing discomfort.

Further, in an implementation, the second housing member 108 of the housing unit 102 is configured to adapt to a target shape associated with the temple region 142 of the user 101, i.e., the second housing member 108 is configured to adapt/conform to a contour of the temple region 142 of the user 101. In an exemplary embodiment, the second housing member 108 is formed of a flexible material that is configured to conform to the contour of the temple region 142 of the user 101 when worn. In an exemplary embodiment, the second housing member 108 may include one or more ridges (or hinge regions) that enable a controlled degree of bending of the second housing member 108, thereby allowing the second housing member 108 to conform to the shape of the temple region 142 of the user 101. It is to be appreciated that the embodiments described above are exemplary and non-limiting and should not be interpreted in any manner to limit the scope of the present disclosure, and any suitable flexible material or structural feature that enables the second housing member 108 to conform to the temple region 142 of the user 101 should be understood to fall within the scope of the present disclosure.

Further, in a preferred embodiment of the present disclosure, the first housing member 106 and the second housing member 108 may be configured to generate a housing cavity (not shown in FIGs) for housing the computing unit 104. It is to be noted that the term “housing cavity” may refer to a hollow, enclosed space formed between the first housing member 106 and the second housing member 108 for holding/accommodating the computing unit 104 of the temple mounted wearable device 100.

Further, the second housing member 108 may include at least the inner layer 114 and the outer layer 116. It is to be noted that the inner layer 114 may refer to a surface or portion located on the inner side of the second housing member 108 which, in combination with the first housing member 106, contributes to defining or generating the housing cavity configured to receive and encapsulate the computing unit 104 and/or any other internal components of the temple mounted wearable device 100. Further, in an exemplary embodiment of the present disclosure, the second housing member 108 may be configured to enable structural support and an interior surface for securing the computing unit 104 within the housing cavity of the temple mounted wearable device 100. In certain embodiments, the second housing member 108 may include one or more slots, recesses, retaining features, and/or engagement structures that are specifically designed to hold, position, or mechanically stabilize the computing unit 104 in place during operation and user 101 movement.

As used herein, the outer layer 116 may refer to an outer side of the second housing member 108 that is exposed to the environment/comes into contact with the skin of the user 101. Further, the outer layer 116 may include at least a set of protrusions 110 and the sticking component (that may be a detachable or fixed component) (not shown in figures). It is to be noted that the term “set of protrusions 110” may refer to one or more projections that stick out from the outer layer 116 of the second housing member 108.

Further, it is to be noted that the term “sticking component” may refer to a mechanism utilized to attach/stick the temple mounted wearable device 100 on the skin of the user 101 such as an adhesive patch, a medical grade tape, a vacuum-based sticking component and any other such like mechanism/combination of mechanisms that may be apparent to a person skilled in the art to implement the present solution. Further, in an exemplary embodiment of the present disclosure, the sticking component may be configured to secure the temple mounted wearable device 100 to the temple region 142 of the user 101.

In an exemplary embodiment of the present disclosure, the sticking component may refer to any component, structure, or attachment mechanism configured to generate a negative pressure or a suction force sufficient to temporarily hold or secure the temple mounted wearable device 100 against the temple region 142 of the user 101. In this context, the term “secure” may refer to temporarily affixing or attaching the temple mounted wearable device 100 to the user's 101 temple region 142 through the use of such sticking component and/or through the generation of a negative G-force effect, thereby providing a stable, comfortable, and reliable fit during usage of the temple mounted wearable device 100. The sticking component may be implemented using any suitable negative-pressure-based technique, for example, micro-suction arrays, vacuum-assisted pads, suction-cup-like structures, one adhesive patch, medical grade tapes or other pressure-differential-generating mechanisms capable of maintaining adequate adhesion without causing discomfort to the user 101.

In another embodiment of the present disclosure, the temple mounted wearable device 100 may be secured to the temple region 142 of the user 101 using a band-based attachment mechanism. In this embodiment, the temple mounted wearable device 100 may be integrated with or coupled to a band configured to extend around at least a portion on the head of the user 101 so as to maintain the temple mounted wearable device 100 in a stable position over the temple region 142. The band may be implemented using any suitable flexible, semi-rigid, or adjustable material that conforms to the contour of the head while providing sufficient retention force to ensure consistent skin-device contact during operation. The band may further include one or more adjustment features, such as tensioning elements, elastic segments, or fastening components, that allow the user 101 to customize the fit to achieve secure placement of the temple mounted wearable device 100. In this embodiment, the band-based attachment mechanism may serve as an alternative to the sticking component described elsewhere in the present disclosure, while still enabling the temple mounted wearable device 100 to overlie the superficial temporal artery (STA) and adjacent vascular structures to implement the solution of the present disclosure.

Further, the second housing member 108 may include the aperture 118. It is to be noted that the “aperture 118” may refer to an opening in the second housing member 108 via which the sensor unit 120 housed within the housing cavity of the temple mounted wearable device 100 may get exposed to the environment/comes into contact with the skin of the user 101 such as the skin overlying the superficial temporal artery (STA) at the temple region 142.

Furthermore, the temple mounted wearable device 100 may include the computing unit 104 installed within the housing unit 102, wherein the computing unit 104 may refer to a set of component(s)/unit(s) configured for processing, analysing, receiving and/or monitoring the set of health attributes of the user 101. Further, in an exemplary embodiment of the present disclosure, the computing unit 104 may include the sensor unit 120 configured to monitor the set of physiological data associated with the user 101. Further, in an exemplary embodiment of the present disclosure, the sensor unit 120 may monitor/receive the set of physiological data associated with the user 101. The set of physiological data may include at least one of the set of photoplethysmography (PPG) data, a set of core body temperature data and the set of inertial measurement unit (IMU) data, and wherein the set of IMU data includes one or more of a set of first IMU data associated with a head region of the user 101, and a set of second IMU data associated with one or more regions below the head region of the user 101. In an implementation, the temple-mounted wearable device 100 may include blue-light, infrared (IR), ultraviolet (UV), and red-green-blue (RGB) emitting light-emitting diodes (LEDs) to monitor the set PPG data.

As used herein, the term “head region” refers to an anatomical region of the user 101 including the entire portion of the body located above the shoulders, including at least the neck and the head of the user 101. The head region, therefore, encompasses structures such as the skull, face, jaw, forehead, temple region 142, and the cervical portion of the neck.

As used herein, the term “regions below the head region” refers to anatomical regions of the user 101 located at or below the shoulders, including at least the shoulders and one or more body parts positioned below the shoulders, such as the torso, arms, hands, legs, or any other body region of the user 101 situated inferior to the head region.

Additionally, in an exemplary embodiment of the present disclosure, as mentioned above, the sensor unit 120 may include the at least one optical sensor 120A and the at least one inertial measurement unit (IMU) sensor 120B. In an exemplary implementation the at least one optical sensors 120A may be the PPG sensor. It is to be noted that the “PPG sensor” may refer to one or more sensor modules/sensor units that detect the set of PPG data associated with the user 101, wherein the set of PPG data may include, for example, a heart rate data, a heart rate variability data, a blood oxygen saturation (SpO2) data, etc. It is to be noted that the “IMU sensor” may refer to one or more sensor modules/sensor units that detect the set of IMU data associated with the user 101, wherein the set of IMU data may include at least an orientation data and a movement data associated with various regions of the user 101 body. It is to be noted that the “orientation data” may indicate a specific physical pose/posture/position of a particular region of the user 101 body. Further, it is to be noted that the “movement data” may indicate a rate of change in position of a particular region of the user 101 body and may include data such as, but not limited to, acceleration, velocity, etc. Further, in an exemplary embodiment of the present disclosure, the IMU sensor may utilize at least one of an accelerometer sensor, a gyroscope sensor and a magnetometer sensor to monitor the set of IMU data associated with the user 101.

Additionally, in an exemplary embodiment of the present disclosure, the set of IMU data may include the set of first IMU data associated with the head region of the user 101, and the set of second IMU data associated with the one or more regions below the head region of the user 101. In such exemplary embodiment, the set of first IMU data may indicate current information/change that relates to a movement, a rotation, an orientation associated with the head region of the user 101 such as eyes, forehead, chin, jaw, lips, head tilt, head nods, etc. Further, in such exemplary embodiment, the set of second IMU data may indicate current information/change that relates to information associated with at least a movement, a rotation and an orientation associated with any region other than the head region of the user 101 i.e., one or more body part below the head region of the user 101 such as hands, shoulders, legs, etc.

It will be appreciated that the sensor unit 120 is not limited to the at least one optical sensor 120A (such as the PPG sensor) and the at least one inertial measurement unit (IMU) sensor 120B. In various embodiments, the sensor unit 120 may additionally or alternatively include any other suitable sensing elements used for monitoring the health and fitness of a user, such as electroencephalography (EEG) sensors, electrocardiography (ECG) sensors, galvanic skin response (GSR) sensors, skin-temperature sensors, blood-oxygen (SpO₂) sensors, bio-impedance sensors, or similar physiological or environmental sensors, without departing from the scope of the present disclosure.

Further, in an embodiment, the computing unit 104 may include the insulation layer 122, wherein the insulation layer 122 is configured to insulate the user 101 from a set of electromagnetic (EM) waves associated with the temple mounted wearable device 100. Furthermore, in an exemplary embodiment of the present disclosure, to insulate the user 101, the insulation layer 122 may be configured to absorb a set of waves associated with the temple mounted wearable device 100. In an exemplary implementation, the insulation layer 122 may be configured to insulate the user 101 from the EM waves that are in a frequency range 2402 MHz to 2480 MHz. In another embodiment of the present disclosure, the housing unit 102 may include the insulation layer 122 configured to absorb the set of waves associated with the temple mounted wearable device 100. In such exemplary embodiments of the present disclosure, the set of waves may include one or more electromagnetic (EM) waves that may be generated by the computing unit 104 and/or one or more components of the computing unit 104 during its operations. It is to be noted that the EM waves may be generated during the operation of the temple mounted wearable device 100, such as communication operations, computing/processing operations and any other such operations.

In a preferred implementation of the present solution, the insulation layer 122 may be configured to absorb the EM waves generated by the one or more electronic components/units/modules such as the computing unit 104, the battery unit 126, etc., of the temple mounted wearable device 100 such that an intensity of the one or more EM waves is eradicated and/or reduced to an acceptable level. Furthermore, in another implementation, the insulation layer 122 may be configured to absorb one or more waves from the set of waves that fall within a predefined wavelength or frequency range. The predefined wavelength or frequency range may correspond to waves that could interfere with or interrupt the operation(s) of other devices in proximity to the temple mounted wearable device 100, or waves that may otherwise pose a potential risk or cause discomfort or harm to the user 101 of the temple mounted wearable device 100.

Additionally, in an exemplary embodiment of the present disclosure, the EM waves may generate noise and/or motion-induced artifacts in the set of physiological data received/monitored by the sensor unit 120, thereby instigating errors in the set of health attributes of the user 101. Further, in such exemplary embodiment, the temple mounted wearable device 100 may be configured to generate a set of enhanced physiological data based on compensating for the noise and/or motion-induced artifacts generated due to EM waves.

Further, the computing unit 104 includes at least the printed circuit board (PCB) 124 configured to process the set of physiological data. The PCB 124 may refer to a rigid and/or flexible insulating substrate with conductive copper traces etched onto it to physically support and electrically connect various units/components of the temple mounted wearable device 100. Additionally, in such exemplary embodiment, the PCB 124 may include at least one or more of the transceivers 128, the processor 130, the charging unit 132 and the memory 134.

In an exemplary embodiment of the present disclosure, the PCB 124 may be formed of a flexible insulating substrate. In such an embodiment, the PCB 124 may be configured to bend, conform, or otherwise adapt to the curvature of the temple mounted wearable device 100 while continuing to physically support and electrically interconnect the transceiver 128, the processor 130, the charging unit 132, the memory 134, and/or other components disposed thereon. In yet another exemplary embodiment of the present disclosure, the PCB 124 may be adapted to include one or more folds, bends, or creases. In this embodiment, the PCB 124 may be arranged in a folded configuration to optimize spatial utilization of the temple mounted wearable device 100, while maintaining electrical connectivity between the transceiver 128, the processor 130, the charging unit 132, the memory 134, and/or other components supported by the PCB 124.

It is to be noted that the transceiver 128 may be configured to perform a set of communication operations associated with the temple mounted wearable device 100 via at least one of a wired mechanism, a wireless mechanism and a combination thereof. It is to be noted that the term “set of communication operations” may refer to one or more method/mechanism for transmitting and receiving information associated with the temple mounted wearable device 100. Furthermore, in an exemplary embodiment of the present disclosure, the transceiver 128 may transmit from the temple mounted wearable device 100, the set of physiological data associated with the user 101 to the user device 140 and may receive from the user 101 via the user device 140, the user 101 feedback associated with the set of physiological data of the user 101.

Further, in an exemplary embodiment of the present disclosure, the processor 130 may analyse the set of physiological data associated with the user 101 for removing/filtering the noise and/or motion-induced artifacts from the set of physiological data. Also, in an exemplary embodiment of the present disclosure, the processor 130 may analyse the user 101 feedback associated with the set of physiological data for enhancing the set of physiological data associated with the user 101 based on the user 101 feedback.

The charging unit 132 may refer to a component that may be configured to manage, regulate, and optimize the transfer of power/energy to the battery unit 126 from an external power system/source. Additionally, in an exemplary embodiment of the present disclosure, the computing unit 104 may include the battery unit 126. In an exemplary embodiment, the charging unit 132 may further include a pogo-pin-based wireless charging interface configured to enable reliable, low-resistance electrical coupling of the temple mounted wearable device 100 with an external charging dock or a cradle. The pogo pins may provide a spring-loaded, self-aligning contact mechanism that ensures consistent power transfer even when the temple mounted wearable device 100 is positioned with minor angular or spatial deviations. Additionally, the pogo-pin arrangement may support sealed or semi-sealed housing designs, thereby improving moisture resistance and overall robustness of the temple mounted wearable device 100.

It should be understood that the description of the wireless charging interface and the associated arrangement as discussed above for transferring power to the temple mounted wearable device 100 is merely exemplary in nature and is not intended to limit the scope of the present disclosure. The use of pogo pins, spring-loaded contacts, or any particular mechanical or electrical configuration for establishing a charging interface shall not be construed as essential unless expressly indicated in the present disclosure. Other charging mechanisms, including but not limited to inductive charging, magnetic connectors, conductive pads, or hybrid interfaces, may be employed without departing from the spirit or scope of the embodiments described herein. All such variations, modifications, and functional equivalents that may be appreciated by a person skilled in the art are intended to fall within the scope of the present disclosure.

The battery unit 126 may refer to a component configured to store the energy/power of the temple mounted wearable device 100. Further, in an exemplary embodiment of the present disclosure, the battery unit 126 may be a compact, lightweight and/or a flexible energy/power storage unit. Also, the battery unit 126 may utilize lithium-ion (Li-ion) or lithium-polymer (LiPo) composition to enable high energy density, better capacity (mAh/Ah), longer cycle life, longer calendar life, faster charging speed, enhanced safety and thermal stability.

The memory 134 may refer to a database configured to store the data associated with the user 101 of the temple mounted wearable device 100. In an exemplary embodiment of the present disclosure, the memory 134 may store the set of physiological data associated with the user 101 received in real-time. Also, the memory 134 may continuously update the stored set of physiological data based on a real-time set of physiological data associated with the use and a set of data updating rules.

In an exemplary embodiment of the present disclosure, the set of data updating rules may trigger a set of instructions to replace the stored set of physiological data in the memory 134 with the real-time set of physiological data associated with the user 101. The set of data updating rules may include a rule that replaces the corresponding portion of the stored set of physiological data with the real-time set of physiological data upon receipt in the memory 134, for example, for data associated with a particular period of time, such as data more than 30 days old. The set of data updating rules may further include a rule that updates the stored set of physiological data in the memory 134 at a predefined periodic interval, such as weekly and/or any other periodic interval that may be apparent to a person skilled in the art. In said rule, at the expiration of each periodic interval, the memory 134 may refresh the stored set of physiological data using the real-time set of physiological data received during a last corresponding time interval, such as replacing, on a particular day T (for example, Monday), a set of physiological data of T-2 (for example, Saturday) with a set of physiological data received during T-1 (for example, Sunday).

Further, in an exemplary embodiment of the present disclosure, the temple mounted wearable device 100 may include the one or more vents defined by at least the set of protrusions 110, and in combination with the sticking component. It is to be noted that the “one or more vents” may refer to a mechanism/element of the temple mounted wearable device 100 that may be utilized for minimizing/reducing sweat factor/level between the second housing member 108 and the skin of the user 101. Further, it would be appreciated by a person skilled in the art that the sweat factor/level may produce noise in receiving/sensing the set of physiological data associated with the user 101. The one or more vents may facilitate reducing/minimizing the noise produced due to the sweat factor/level. Furthermore, the one or more vents may facilitate longer use of the temple mounted wearable device 100 based on maintaining optimal device temperatures, which significantly boosts battery efficiency and longevity by enabling stable chemical reactions and minimizing degradation of the battery unit 126. In an exemplary embodiment of the present disclosure, the one or more vents may allow the movement of at least air and sweat between the outer layer 116 of the second housing member 108 and the skin of the user 101 to reduce/minimize the level of sweat produced in the region where the temple mounted wearable device 100 is affixed to the user 101 body.

Additionally, in an exemplary embodiment of the present disclosure, the housing unit 102 may include the housing assembly member 112, wherein the housing assembly member 112 is based on at least one of an interlocking technique and an adhesive technique. In such exemplary embodiment, the housing assembly member 112 may refer to a component/unit utilized for joining/attaching the first housing member 106 and the second housing member 108. Further, it is to be noted that the term “interlocking technique” may refer to one of a method, a component, a material and a mechanism for joining/attaching at least the first housing member 106 and the second housing member 108. Furthermore, it is to be noted that the term “adhesive technique” may refer to a mechanism/method for joining/attaching at least the first housing member 106 and the second housing member 108 via one or more adhesive materials such as, but not limited to, epoxy resin, polyvinyl acetate (PVA) white glue, rubber, etc.

Further, in an exemplary embodiment of the present disclosure, the first housing member 106 and the second housing member 108 may be joined/attached together based on one or more fusing techniques, such as, but is not limited to, a heat-activated fusing technique, a thermal bonding technique, etc. In such one or more fusing techniques, controlled amounts of heat and pressure may be applied for joining/attaching the first housing member 106 and the second housing member 108. Furthermore, in exemplary embodiment of the present disclosure, the housing assembly member 112 may utilize a mechanism, a component and/or a material for maintaining a water resistant rating of the temple mounted wearable device 100 while allowing the temple mounted wearable device 100 to adapt to the shape of the target region of the user 101 such as the temple region 142 for e.g., by providing flexibility in the second housing member 108 to match the contour of the user's 101 target region.

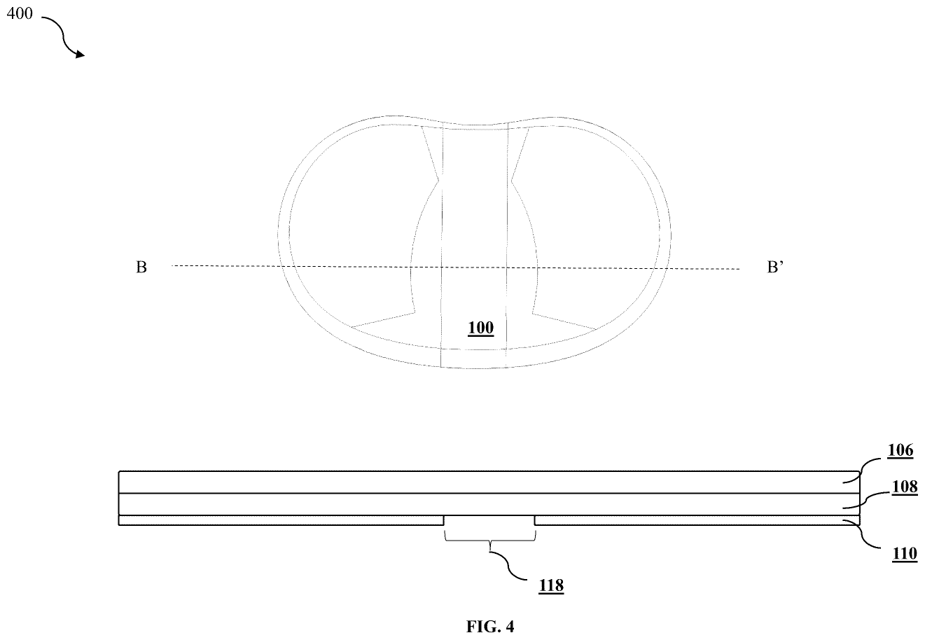

FIG. 4 illustrates an exemplary cross-section view 400 of the temple mounted wearable device 100, according to an embodiment of the present disclosure. In an embodiment, the cross-section of the temple mounted wearable device 100 is illustrated along the line B-B′ as depicted. As shown, a portion of the set of protrusions 110 is cut out so that the at least one optical sensor 120A may emit and receive the optical signals via the aperture 118 from the cut out.

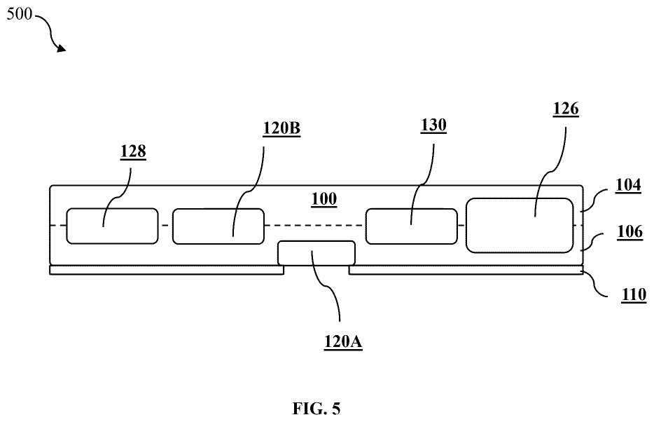

FIG. 5 illustrates another exemplary cross-sectional view 500 of the temple mounted wearable device 100, according to an embodiment of the present disclosure. As shown, the temple mounted wearable device 100 includes the housing unit 102, including the first housing member 106 and the second housing member 108. The second housing member 108 includes the aperture 118 configured to allow the sensor unit 120 to interface with the temple region 142 of the user 101.

The sensor unit 120 includes the at least one optical sensor 120A and the at least one inertial measurement unit (IMU) sensor 120B, each mounted on the printed circuit board (PCB) 124 positioned within the housing unit 102. In an exemplary implementation, the at least one optical sensor 120A may be oriented towards the aperture 118 to emit and receive optical signals from the temple region 142, while the at least one IMU sensor 120B may be positioned to detect motion and orientation attributes associated with the user 101. Further, the sensor unit 120 may be configured to contact the temple region 142 through the aperture 118.

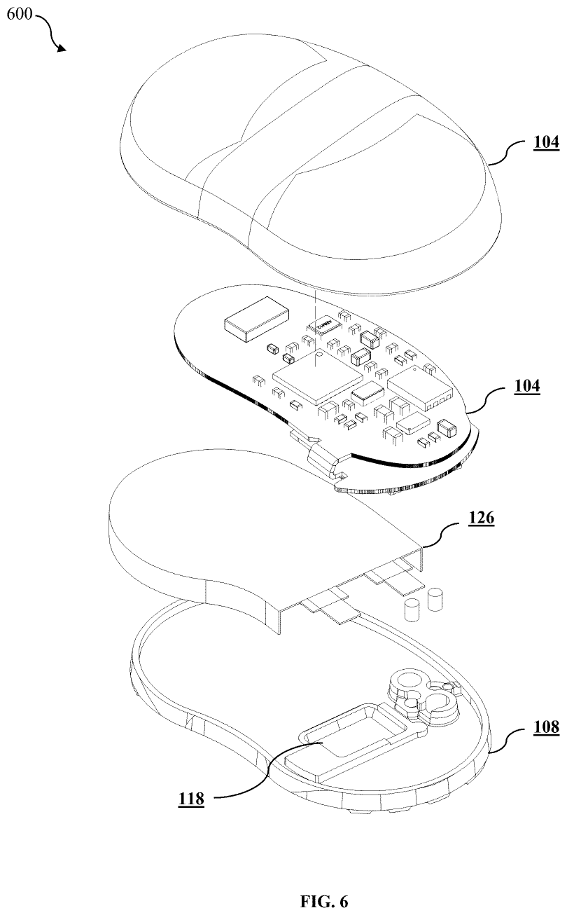

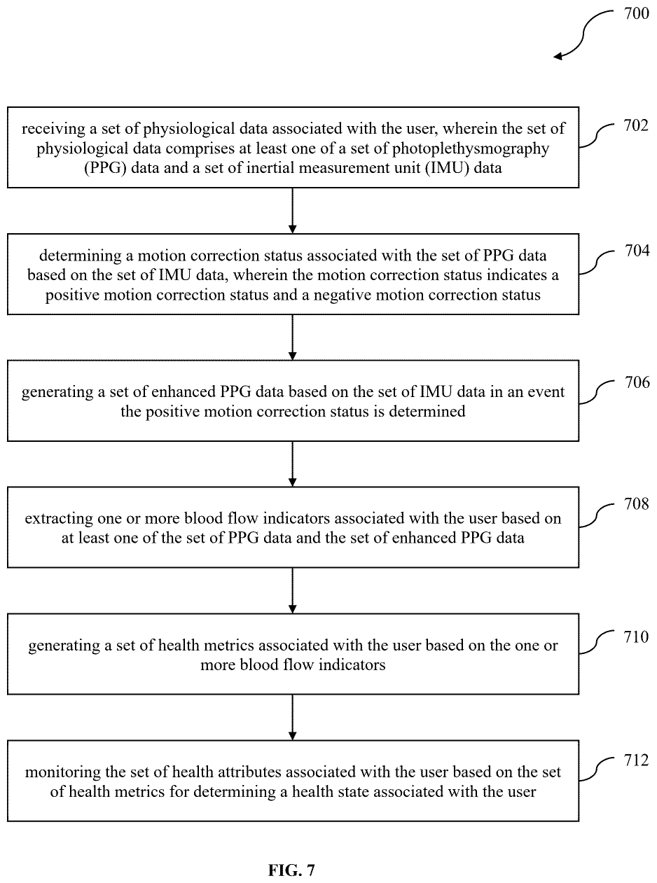

FIG. 6 depicts an exemplary exploded isometric view of the temple mounted wearable device 100, according to an embodiment of the present disclosure. In an embodiment, the first housing member 106 may be fixed atop the second housing member 108 to compose the housing unit 102 and thereby define the housing cavity. In an implementation, an adhesive layer may be affixed to a bottom portion of the second housing member 108 of the housing unit 102. Further, in an embodiment, the second housing member 108 and the adhesive layer may have at least a portion cutout, to affix the sensor unit 120, such as the at least one optical sensor 120A, to the outer surface of the second housing member 108 of the housing unit 102. In an embodiment, the portion cutout of the adhesive layout may allow one or more optical signals generated by the sensor unit 120 to be transmitted towards the skin of the user 101. Further, as depicted in FIG. 4, a portion of the adhesive layer is cut out so that the sensor unit 120 may emit and receive the one or more optical signals via the aperture 118.

The computing unit 104 is disposed within the housing cavity formed by the first housing member 106 and the second housing member 108 (also depicted in FIG. 6). The computing unit 104 includes at least the processor 130, the memory module 134, the transceiver 128, and the charging unit 132 mounted on the PCB 124. A battery unit 126 is positioned adjacent to the PCB 124 to supply power to the processor 130, the sensor unit 120, and the transceiver 128. The insulation layer 122 is provided within the housing unit 102 to insulate the user 101 from the set of electromagnetic (EM) waves/signals associated with the temple mounted wearable device 100.

The second housing member 108 further includes at least the inner layer 114 and the outer layer 116. The outer layer 116 includes the set of protrusions 110, and the sticking component is configured to secure the temple mounted wearable device 100 to the temple region 142 of the user 101. The one or more vents may be defined by the set of protrusions 110 and the sticking component to facilitate airflow and improve comfort during prolonged usage.

In an embodiment, the temple mounted wearable device 100 may further include a haptic actuator (not shown in the figure) configured to provide haptic feedback to the user 101 based on one or more health-related notifications. In another embodiment, the temple mounted wearable device 100 may include a touch sensor (not shown in the figure) disposed on the first housing member 106 to receive one or more input commands from the user 101.

The temple mounted wearable device 100 may be secured to the user 101 using the sticking component of the outer layer 116 or by alternate attachment mechanisms such as a mechanical coupling, a magnetic interface, a nano-pressure microstructure, or a vacuum sealing interface. In an embodiment, the sticking component or adhesive layer is replaceable and may be replaced after a predetermined number of days of usage.

In one embodiment, the temple mounted wearable device 100 may be a low-mass, miniature structure. The temple mounted wearable device 100 may have dimensions of 2.42+/−0.02 cm ×1.55+/−0.02 cm, and may weigh between approximately 1.7 grams and 2.2 grams.

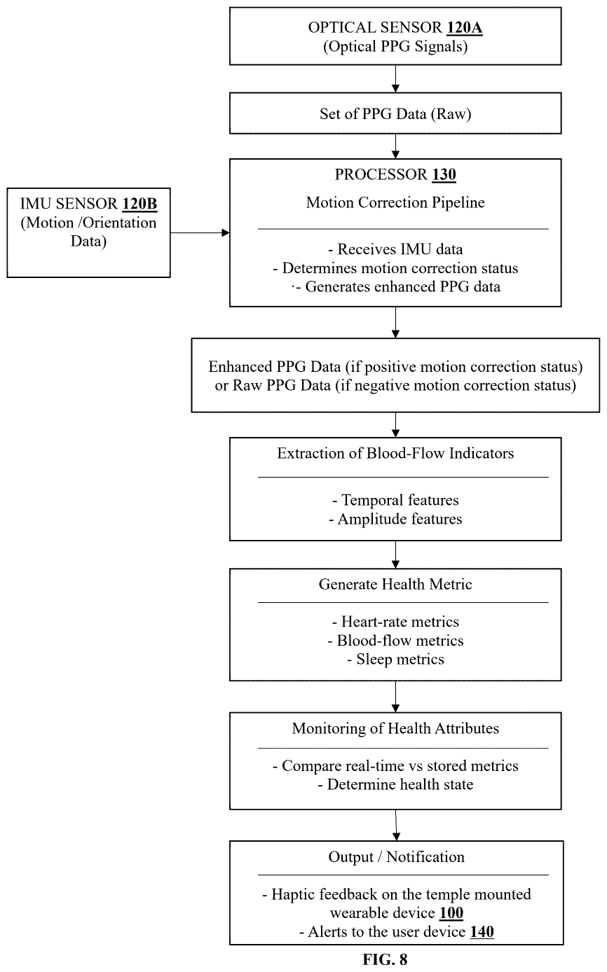

Now referring to FIG. 7 illustrates an exemplary flow diagram of a method 700 for monitoring a set of health attributes associated with the user 101, in accordance with the exemplary embodiments of the present disclosure. In an implementation, the method 700 is implemented by the temple mounted wearable device 100. In an implementation of the present disclosure, the processor 130 is configured to implement the method 700 as disclosed herein.

At step 702, the method 700 includes receiving the set of physiological data associated with the user 101, wherein the set of physiological data may include at least one of the set of photoplethysmography (PPG) data and the set of inertial measurement unit (IMU) data. In an embodiment of the present disclosure, the set of physiological data may be detected from the superficial temporal artery (STA) associated with the temple region 142 of the user 101. Further, the sensor unit 120 of the temple mounted wearable device 100 may be configured to sense the set of physiological data associated with the user 101. Further, in an exemplary embodiment of the present disclosure, the sensor unit 120 may include the at least one optical sensor 120A (such as the PPG sensor) and the at least one inertial measurement unit (IMU) sensor 120B and a combination thereof.

In an exemplary embodiment of the present disclosure, the PPG sensor may utilize one or more PPG data monitoring techniques to monitor the set of PPG data of the user 101. In an implementation, an exemplary PPG data monitoring technique may employ a set of light emitting diodes (LEDs) and photodiodes to monitor the set of PPG data. The LEDs may include at least one red (R), one green (G), and one infrared (IR) LED configured to illuminate a capillary bed of the user 101 with light signals of differing wavelengths. The photodiodes may thereafter receive the light signals reflected, transmitted, or scattered from the capillary bed.

In such an embodiment, the PPG data monitoring technique may correlate variations in reflected light intensity associated with wavelengths of the R, G, and IR LEDs to determine the set of PPG data. It is to be noted that each wavelength may exhibit a distinct absorption and reflection profile based on blood volume changes within the underlying tissue, thereby enabling the sensor unit 120 to monitor the set of PPG data.

In another embodiment, the PPG data monitoring technique may include a calibration mechanism configured to adjust the intensity of the LEDs based on one or more user 101 parameters. These parameters may include, but are not limited to, skin tone, tissue density, ambient lighting conditions, and a detected signal-to-noise ratio associated with the reflected light. Based on these parameters, the computing unit 104 may dynamically increase or decrease the LED intensity to optimize penetration depth, improve reflectance quality, and enhance PPG data accuracy. Such a calibration mechanism may also adjust the LED intensity based on a detected sweat level at the region of application of the temple mounted wearable device 100. In another exemplary embodiment of the present disclosure, the calibration mechanism may adjust the LED intensity based on a change in the distance of contact between the temple mounted wearable device 100 and the temple region 142, wherein such a change in the distance of contact may occur due to factors such as tape ageing or any other variation in the adhesion characteristics of the sticking component. In an exemplary implementation, the calibration mechanism may iteratively evaluate reflected light values received by the photodiodes and may adjust the LED drive current until a predefined signal-quality threshold is achieved. Such adaptive calibration may ensure consistent PPG signal acquisition across different users and usage conditions, thereby improving the reliability of the set of physiological data monitored by the temple mounted wearable device 100.

Further, in an exemplary embodiment of the present disclosure, prior to receiving the set of PPG data, the temple mounted wearable device 100 may utilize a first trained machine learning (ML) model (also referred to as the first trained model) for dynamically altering a set of sensor parameters associated with the at least one optical sensor 120A to optimise quality of the received set of PPG data based on at least one of skin tone, usage pattern of the user 101, or a predefined signal-quality threshold.

As used herein, the term “quality of the received set of PPG data” may refer to a measure of the reliability, clarity, and usability of the PPG signals obtained from the at least one optical sensor 120A such as the PPG sensor, based on one or more signal attributes such as amplitude stability, signal-to-noise ratio, waveform morphology, baseline consistency, and the absence of motion artifact induced distortions. The quality of the received set of PPG data may indicate the extent to which the captured signals are suitable for accurate extraction of physiological parameters associated with the user 101.

The set of sensor parameters may include at least one of a sampling rate parameter, a pulse wave intensity parameter, a pulse duration parameter, a power parameter (e.g., wavelength), and an integration time parameter. In an exemplary embodiment, the first trained model may also alter the intensity of light emitted by the LEDs based on user-specific parameters such as skin tone or the distance of the capillary bed from the at least one optical sensor 120A.

Further, it is to be noted that “IMU sensor” may refer to one or more sensor modules/sensor units that monitor the set of IMU data associated with the user 101, wherein the set of IMU data may include at least an orientation data and a movement data associated with various regions of the user 101 body. Further, in an embodiment of the present disclosure, the IMU sensor may utilize at least one of an accelerometer sensor, a gyroscope sensor and a magnetometer sensor to monitor the set of IMU data. It is to be noted that the “orientation data” may indicate a specific physical pose/posture/position and/or a directional change associated with a particular region of the user 101 body. As used herein, the “movement data” may indicate a rate of change in position of a particular region of the user 101 body and may include data such as, but is not limited to, acceleration, velocity, etc.

For ease of understanding, consider an example wherein a user A is wearing a wearable device X. The user A moves their head from position 1 to position 2. Herein, the orientation data of the user A may include that at time T1 the position of the head of the user A was position 1 and at time T2 the position of the head of the user A was position 2, whereas the movement data of the user A may include at least a velocity and an acceleration at which the head of the user A moved from the position 1 to position 2.

It is to be noted that the abovementioned sensors are only exemplary in nature and in no manner intended to limit the scope of the present disclosure. The sensor unit 120 may include any other sensor(s) as may be appreciated by the person skilled in the art to implement the present solution.

Further, in an exemplary embodiment of the present disclosure, the set of IMU data may include the set of first IMU data associated with the head region of the user 101, and the set of second IMU data associated with the one or more regions below the head region of the user 101. It is to be noted that the “set of first IMU data” may indicate current information/change related to a movement, a rotation and an orientation associated with the head region of the user 101, such as eyes, forehead, chin, jaw, lips, head tilt, head nods, etc. Further, it is to be noted that the set of second IMU data may indicate current information/change related to a movement, a rotation and an orientation associated with any region/body part, other than the head region of the user 101 i.e., one or more body part below the head region of the user 101 such as hands, shoulders, legs, etc.

Next, at step 704, the method 700 includes determining a motion correction status associated with the set of PPG data based on the set of IMU data, wherein the motion correction status indicates one of a positive motion correction status and a negative motion correction status. It is to be noted that the term ‘motion correction status’ may indicate whether motion correction is required for the set of PPG data, based on an assessment of the set of IMU data that reflects the presence or absence of motion-induced artifacts. The term ‘positive motion correction status’ as used herein may indicate that motion correction (e.g., filtering or compensating for motion-related noise) is required, whereas a ‘negative motion correction status’ may indicate that such correction is not required. The level or presence of motion-induced artifacts may be determined based on the set of IMU data. Furthermore, in an exemplary embodiment of the present disclosure, the motion correction status associated with the set of PPG data may be determined based on one or more IMU data from the set of IMU data, i.e. the one or more IMU data associated with at least one of the set of first IMU data and the set of second IMU data.

In an exemplary embodiment of the present disclosure, prior to determining the motion correction status associated with the set of PPG data, the method 700 may include filtering the set of PPG data and the set of IMU data from the set of physiological data based on one or more data filtering techniques. In such exemplary embodiment, the one or more data filtering techniques may include a band pass filtering technique, a low-pass filtering technique, feature-based filtering technique and such other data filtering techniques.

It is to be noted that the abovementioned data filtering techniques are only exemplary in nature and in no manner intended to limit the scope of the present disclosure. The one or more data filtering techniques may include any other technique(s) as may be appreciated by the person skilled in the art to implement the present solution.

Further, in an exemplary embodiment of the present disclosure, the method 700 may include determining a temporal alignment status associated with the set of IMU data and the set of PPG data. As used herein, the term ‘temporal alignment status’ may refer to an indicator representing the synchronization accuracy between a timestamp associated with the set of PPG data (hereinafter also referred as a set of PPG data time-period values associated with the set of PPG data) and a timestamp associated with the set of IMU data (hereinafter also referred as a set of IMU data time-period values associated with the set of IMU data). In such an exemplary embodiment, the temporal alignment status is one of a positive temporal alignment status or a negative temporal alignment status.

Further, it is to be noted that the positive temporal alignment status associated with the set of IMU data and the set of PPG data may be determined in an event the set of IMU data time-period values associated with the set of IMU data is equal to the set of PPG data time-period values associated with the set of PPG data. Further, it is to be noted that the negative temporal alignment status associated with the set of IMU data and the set of PPG data may be determined in an event the set of IMU data time-period values associated with the set of IMU data differ from the set of PPG data time-period values associated with the set of PPG data. Further, it is to be noted that the set of IMU data time-period values may refer to one or more specific durations of time during which the set of IMU data is received (i.e., timestamps associated with the IMU data). Further, the set of PPG data time-period values may refer to one or more specific durations of time during which the set of PPG data is received (i.e., timestamps associated with the PPG data).

Thereafter, the method 700 may include determining, in the set of physiological data, a set of IMU weights associated with the set of IMU data in an event the positive temporal alignment status is determined. It is to be noted that the set of IMU weights may refer to one or more values indicating a level of contribution/relevance of the set of IMU data in the set of physiological data. Further, in an exemplary embodiment, the lower the value of the set of IMU weights of the set of IMU data in the set of physiological data, the lower the relevance/contribution of the set of IMU data in the set of physiological data.

For ease of understanding, consider an example wherein the set of IMU weights ranges from 0 to 10, with 0 indicating a lower contribution of the set of IMU data to the set of physiological data and 10 indicating a higher contribution. In a first event, if an IMU weight is determined to be 3, and in a second event the IMU weight is determined to be 6, the contribution of the set of IMU data to the set of physiological data in the first event is lower than in the second event.

Further, in an exemplary embodiment of the present disclosure, the method 700 may include determining the motion correction status associated with the set of PPG data based on the set of IMU weights. It is to be noted that the positive motion correction status associated with the set of PPG data is determined when one or more weight values in the set of IMU weights are either equal to a first predefined threshold value or above the first predefined threshold value. For example, if the first predefined threshold value is 0.7 and one of the weight values in the set of IMU weights is 0.7 or 0.8, the positive motion correction status may be determined for the corresponding set of PPG data. It is to be noted that the term “first predefined threshold value” may refer to a pre-configured/predetermined limit of the set of IMU weights associated with the set of IMU data.

For ease of understanding, consider an example wherein the first predefined threshold value for a user A is 3. If the IMU weight associated with a particular IMU data of user A in a received set of physiological data is 3 or higher (e.g., 3.5, 4, 4.5, etc.), a positive motion correction status associated with the corresponding set of PPG data may be determined. If the IMU weight associated with said particular IMU data of user A in the received set of physiological data is below 3 (e.g., 2.8, 2.5, 2, etc.), a negative motion correction status associated with said corresponding set of PPG data may be determined.

Further, at step 706, the method 700 includes generating a set of enhanced PPG data based on the set of IMU data in an event that the positive motion correction status is determined. It is to be noted that the “set of enhanced PPG data” may refer to a PPG data that has been adapted by removing/filtering/compensating the noise and/or motion-induced artifacts from the set of PPG data (i.e., removing the error in the set of PPG data based on the weight/value of the set of IMU data in the set of physiological data). Further, in an exemplary embodiment of the present disclosure, the set of enhanced PPG data may be generated based on removing/filtering/compensating a noise and/or motion-induced artifacts induced in the set of PPG data, wherein such noise and/or motion-induced artifacts are associated with at least one of the set of first IMU data associated with the head region of the user 101, and the set of second IMU data associated with the one or more regions below the head region of the user 101.

As used herein, the term “head region” refers to an anatomical region of the user 101 including the entire portion of the body located above the shoulders, including at least the neck and the head of the user 101. The head region, therefore, encompasses structures such as the skull, face, jaw, forehead, temple region 142, and the cervical portion of the neck.

As used herein, the term “regions below the head region” refers to anatomical regions of the user 101 located at or below the shoulders, including at least the shoulders and the one or more body parts positioned below the shoulders, such as the torso, arms, hands, legs, or any other body region of the user 101 situated inferior to the head region.

Further, in an exemplary embodiment of the present disclosure, the method 700 may include determining, in the set of IMU data, at least one of a set of first IMU weight associated with the set of first IMU data and a set of second IMU weight associated with the set of second IMU data in an event the positive motion correction status is determined. It is to be noted that the term “set of first IMU weight” may refer to one or more values indicating a level of contribution/relevance of the set of first IMU data in the set of IMU data. Further, it is to be noted that the term “set of second IMU weight” may refer to one or more values indicating a level of contribution/relevance of the set of second IMU data in the set of IMU data.

For ease of understanding, consider an example, in which the temple mounted wearable device 100 is worn on the temple region 142 of the user 101 for monitoring one or more IMU data associated with the head region and/or regions below the head of the user 101 during a brisk walk. During this activity, the user 101 frequently turns his head to look around, resulting in higher motion activity in the head region compared to the rest of the body. In an event where a positive motion correction status is determined, the method 700 may compute IMU weights for each region. In this scenario, the set of first IMU weight may be assigned a higher value (for example, 7.2) to indicate that the head-region motion contributes significantly to the overall IMU data. Conversely, the set of second IMU weight may be assigned a lower value (for example, 3.1) to indicate that motion from the regions below the head contributes less to the overall IMU data. These weights help identify which region's motion is primarily responsible for the motion-induced artifacts that need to be compensated for generating the set of enhanced PPG data.

Further, the method 700 may include identifying from the set of IMU data, a set of target IMU data based on the set of first IMU weight and the set of second IMU weight and a second predefined threshold value. It is to be noted that the term ‘set of target IMU data’ may refer to a particular IMU data associated with a target region of the user's 101 body that is relevant for monitoring the set of health attributes of the user 101 based on a set of weights associated with said particular IMU data. In an exemplary embodiment of the present disclosure, the target region may be at least one of the head region or the one or more regions below the head region of the user 101.

Further, it is to be noted that the term ‘second predefined threshold value’ refers to a predetermined limit associated with one or more of the set of first IMU weight and the set of second IMU weight, above which the contribution of the corresponding IMU data is considered sufficiently significant for that IMU data to be identified as the set of target IMU data for motion-correction processing. Further, in an exemplary embodiment of the present disclosure, the set of first IMU data may be identified as the set of target IMU data in an event that a value of the set of first IMU weight in the set of IMU data is one of a value equal to the second predefined threshold value and a value above the second predefined threshold value. Further, the set of second IMU data may be identified as the set of target IMU data in an event where a value of the set of second IMU weight in the set of IMU data is one of a value equal to the second predefined threshold value or a value above the second predefined threshold value.

For ease of understanding, consider an example wherein the second predefined threshold value for a user A is set to 3. In this scenario, if the value of the set of first IMU weight associated with the set of first IMU data of user A is equal to or greater than 3 (for example, 3.0, 3.5, 4.0, etc.), the set of first IMU data may be identified as the set of target IMU data. Similarly, if the value of the set of second IMU weight associated with the set of second IMU data of user A is equal to or greater than 3, the set of second IMU data may be identified as the set of target IMU data. Conversely, if the corresponding IMU weight is below 3 (for example, 2.8, 2.5, 2.0, etc.), that IMU data may not be identified as the set of target IMU data.

Further, the method 700 may include generating the set of enhanced PPG data based on the set of target IMU data. In an exemplary embodiment of the present disclosure, the method 700 may include filtering the set of first IMU data from the set of IMU data in an event that the identified set of target IMU data includes the set of first IMU data.

In an exemplary embodiment of the present disclosure, when the identified set of target IMU data includes at least one of the set of first IMU data and the set of second IMU data, the method 700 may include filtering the set of first IMU data from the set of IMU data for subsequent processing. In such an embodiment, the set of first IMU data may be filtered using one or more IMU data filtering techniques, such as, but not limited to, bandpass filtering, least mean square (LMS) filtering, spectral analysis, or wavelet denoising, to obtain the first IMU data.

In an embodiment, an exemplary IMU data filtering technique may first encompass identifying, in the set of IMU data, the set of first IMU data based on one or more IMU attributes associated with one or more of the set of first IMU data and the set of second IMU data. Once the set of first IMU data is identified, the exemplary IMU data filtering technique may attenuate frequency components of the set of first IMU data that fall outside a motion-relevant frequency range. In another example, a least mean square (LMS) adaptive filtering technique may be employed to iteratively adjust filter coefficients so as to attenuate said frequency components in order to filter the set of first IMU data. In yet another example, a wavelet-based denoising technique may be applied in which the set of first IMU data is decomposed into wavelet coefficients, compared to a predefined frequency value range to attenuate said frequency components in order to filter the set of first IMU data. Thereafter, the filtered set of first IMU data may then be utilized in motion-artifact compensation process for generating the set of enhanced PPG data.

In an embodiment, an exemplary adaptive filtering technique may be employed to further enhance the quality of the set of first IMU data prior to its use in generating the set of enhanced PPG data. In such an embodiment, the adaptive filtering technique may operate by computing, for each iteration, a set of filter weights based on one or more IMU attributes associated with the set of first IMU data and the set of second IMU data. The adaptive filtering technique may then update the set of filter weights in accordance with a predefined rule, such artificial intelligence/machine learning rule, configured to minimize an error function representative of a difference between a predicted motion-artifact component and an estimated motion-artifact component present in the set of first IMU data. By iteratively adjusting the filter weights to minimize said error function, the adaptive filtering technique may attenuate frequency components of the set of first IMU data that fall outside a motion-relevant frequency range. Thereafter, the adaptively filtered set of first IMU data may be utilized in a motion-artifact compensation process for generating the set of enhanced PPG data, in accordance with the embodiments of the present disclosure.

It is to be noted that the abovementioned filtering techniques are only exemplary in nature and in no manner intended to limit the scope of the present disclosure. The filtering techniques may include any other technique(s) as may be appreciated by the person skilled in the art to implement the present solution.

Further, the method 700 may include determining a set of first IMU attributes associated with the set of first IMU data, wherein the set of first IMU attributes includes at least one of a set of first motion attributes and a set of first orientation attributes. In an exemplary embodiment of the present disclosure, the set of first motion attributes may refer to data indicative of one or more physical movements (e.g., acceleration, rotation, velocity, and the like) of the head region of the user 101 during a particular period of time. Similarly, the set of first orientation attributes may refer to data indicative of a relative and/or absolute change in one or more postures or positions of the head region of the user 101 during the particular period of time.

In an exemplary embodiment, the method 700 for determining the set of first IMU attributes associated with the set of first IMU data may include analyzing the set of first IMU data to determine the set of first motion attributes and the set of first orientation attributes from the set of first IMU attributes based on utilizing one or more data processing techniques. In some embodiments, an exemplary data processing technique for determining the set of first IMU attributes may further include performing one or more computational operations, such as feature extraction, threshold evaluation, or pattern identification, on the set of first IMU data to obtain attribute values that characterize the motion and/or orientation of the head region during a particular period of time. Accordingly, the method 700 may determine the set of first IMU attributes in a manner that enables subsequent weighting, fusion, or motion-correction processing using the set of first IMU data.

Further, in another exemplary embodiment of the present disclosure, for determining the set of first IMU attributes, the method 700 may include estimating one or more movements in a set of muscles associated with the head region of the user 101. In such an embodiment, the method 700 may include differentiating between movements and/or orientations associated with the head region of the user 101 based on the one or more movements detected in the set of muscles associated with the head region (i.e., the set of first IMU data) and a set of user historic movement data that indicates different movements associated with the user 101. As used herein, the term “set of user historic movement data” may refer to a set of previously recorded or derived data values that characterize one or more past movements, postures, or orientation patterns associated with the user 101.

Further, the method 700 may include identifying a set of first user activity types associated with the set of first IMU data based on the set of first IMU attributes, wherein the set of first user activity types includes at least one of a facial muscle activity and a non-facial muscle activity. It is to be noted that the “facial muscle activity” may indicate one or more movements in one or more facial muscles in the head region of the user 101, but may not include one or more movements of the head of the user 101. Further, in an exemplary embodiment of the present disclosure, the one or more facial muscle activities may include, but is not limited to, orofacial movements (e.g., Lip pursing, Jaw opening/closing, etc.), eye blinking, etc. Further, it is to be noted that the one or more non-facial muscle activities may indicate one or more movements in one or more body parts associated with the head region of the user 101, such as, but not limited to, head rotation, head nodding, or other head-related movements.

Further, in an exemplary embodiment of the present disclosure, for identifying the set of first user activity types, the method 700 may include analysing the set of first IMU attributes to determine the one or more physical movements and/or the one or more relative changes in the position/posture of the head region of the user 101 based on one or more IMU attributes analysing techniques. In some embodiments, an exemplary IMU attributes analysing technique may identify the set of muscles in the head region associated with the one or more physical movements and/or the one or more relative changes in the position/posture of the head region of the user 101. Thereafter, based on the identified set of muscles in the head region of the user 101, the set of first user activity types may be identified.

Further, in an exemplary embodiment of the present disclosure, the method 700 may include filtering from the set of first IMU data, a set of facial muscle activity IMU data and a set of non-facial muscle activity IMU data based on the set of first user activity types. In an event, if the set of first user activity types is the facial muscle activity, the method 700 may include filtering the set of facial muscle activity IMU data from the set of first IMU data. Further, in another event, if the first user activity type is the non-facial muscle activity, the method 700 may include filtering the set of non-facial muscle activity IMU data from the set of first IMU data. Further, in an exemplary embodiment of the present disclosure, the set of facial muscle activity IMU data is associated with the one or more facial muscles of the user 101, and wherein the set of non-facial muscle activity IMU data is associated with one or more body part movements associated with the head region of the user 101, such as neck movement, etc.

In an embodiment of the present disclosure, the method 700 may include implementing an exemplary filtering technique configured to filter, from the set of first IMU data, one or more of the set of facial muscle activity IMU data and the set of non-facial muscle activity IMU data based on the set of first user activity types. In such an embodiment, the exemplary filtering technique may initially analyze the set of first IMU data to identify frequency-domain and amplitude-domain characteristics associated with the set of first IMU attributes. Further, based on this analysis, the exemplary filtering technique may determine whether the set of first user activity types corresponds to the facial muscle activity or the non-facial muscle activity.

In an event in which the set of first user activity types is determined to be the facial muscle activity, the exemplary filtering technique may include filtering the set of facial muscle activity IMU data from the set of first IMU data. In such an embodiment, the facial muscle activity IMU data may be identified by detecting relatively higher-frequency, lower-amplitude variations in the set of first IMU data over a predefined period of time let's say, 2 seconds, 5 seconds, etc., which may indicate localized movements in one or more facial muscles of the user 101, such as orofacial movements or eye blinking.

Whereas in another event, in which the set of first user activity types is determined to be the non-facial muscle activity, the exemplary filtering technique may include filtering the set of non-facial muscle activity IMU data from the set of first IMU data. In such an embodiment, the non-facial muscle activity IMU data may be identified by detecting relatively lower-frequency, higher-amplitude variations in the set of first IMU data over said predefined period of time that indicate movements of one or more body parts associated with the head region of the user 101, such as head rotation, head nodding, or other head-related movements. Accordingly, in said embodiment, by utilizing the frequency-domain characteristics and the amplitude-domain characteristics associated with the set of first IMU data, the method 700 may selectively filter the set of facial muscle activity IMU data and/or the set of non-facial muscle activity IMU data.

Further, the method 700 may include generating the set of enhanced PPG data based on at least one of the set of facial muscle activity IMU data and the set of non-facial muscle activity IMU data. In an exemplary embodiment of the present disclosure, for generating the set of enhanced PPG data, the method 700 may include fetching a first set of error correction values from a set of predefined error correction values based on the set of facial muscle activity IMU data in an event that the identified set of first user activity types includes the facial muscle activity. It is to be noted that the set of predefined error correction values may refer to one or more predetermined parameters for removing/filtering, from the set of physiological data, the noise and/or motion-induced artifacts caused by the facial muscle activity.

In an exemplary embodiment of the present disclosure, the method 700 may include utilizing a first trained model to generate the set of predefined error correction values based on both historical and current physiological data of the user 101. In such an embodiment, the first trained model may access, from the memory 134 of the temple mounted wearable device 100, a set of user historic movement data including a historic set of IMU data, a historic set of PPG data, one or more patterns associated with historic facial muscle activity and non-facial muscle activity of the user 101, and one or more historic error correction values associated with the historic facial muscle activity. The set of user historic movement data may include characteristic frequency-domain and amplitude-domain signatures corresponding to different types of movements of the head region and the facial muscles of the user 101.

Further, in said exemplary embodiment, the first trained model may analyze a set of current IMU attributes derived from the set of first IMU data to estimate a current movement type associated with the user 101. In one exemplary embodiment, the first trained model may compare the current IMU attributes with the set of user historic movement data to identify a similarity between the current IMU attributes and one or more previously observed movement patterns. Further, based on the comparison, the first trained model may be configured to estimate whether the current movement corresponds to the facial muscle activity or the non-facial muscle activity.

Thereafter, in said exemplary embodiment, the first trained model may generate the set of predefined error correction values based on the estimated movement type and the corresponding patterns stored in the set of user historic movement data. In an event the estimated movement type corresponds to a facial muscle activity, the first trained model may generate error correction values configured to remove or reduce noise and/or motion-induced artifacts caused by the facial muscle activity in the set of physiological data.

Further, in an exemplary embodiment of the present disclosure, the method 700 may include determining a set of non-facial IMU attributes associated with the non-facial muscle activity IMU data in an event that the identified set of first user activity types includes the non-facial muscle activity. It is to be noted that the term ‘set of non-facial activity IMU attributes’ may refer to one or more parameters indicative of movements of one or more body parts associated with the head region of the user 101, such as, but not limited to, orientation or position of the head, angular velocity of the head, linear velocity of head movement, or other head-related motion characteristics.

Further, in an exemplary embodiment of the present disclosure, for determining the set of non-facial activity IMU attributes, the method 700 may include analyzing the non-facial muscle activity IMU data to extract one or more IMU-based characteristics associated with movement of the head of the user 101. The extracted characteristics may include, but are not limited to, time-domain features (such as acceleration values, angular velocity values, linear velocity estimates, or peak amplitude), frequency-domain features (such as dominant frequency components or spectral energy distribution), orientation-domain features (such as tilt, inclination, yaw, pitch, or roll), and statistical features (such as variance, root-mean-square values, or signal entropy). Based on these extracted characteristics, the method 700 may determine the set of non-facial activity IMU attributes. In an exemplary embodiment, the set of non-facial activity IMU attributes may include at least one of a set of non-facial activity motion attributes and a set of non-facial activity orientation attributes. It is to be noted that the term “set of non-facial activity motion attributes” may refer to data indicative of one or more physical movements of the head of the user 101, such as, but not limited to, acceleration, angular velocity, linear velocity, or rotational rate associated with the head movement. As used herein, the term “set of non-facial activity orientation attributes” may refer to data indicative of one or more postures or positions of the head of the user 101, including, but not limited to, tilt, inclination, angular displacement, or other orientation-related characteristics.

Further, in an exemplary embodiment of the present disclosure, the method 700 may include determining in the set of first IMU data, a set of non-facial IMU weights associated with the set of non-facial muscle activity IMU attributes. It is to be noted that the term “set of non-facial IMU weights” may refer to one or more values indicating a level of contribution/relevance of the set of non-facial muscle activity IMU attributes in the set of first IMU data.

Further, in an exemplary embodiment of the present disclosure, the method 700 may include generating the set of second error correction values associated with the non-facial muscle activity based on the set of non-facial IMU weights. It is to be noted that the set of second error correction values may refer to one or more parameters configured to remove or reduce, from the set of physiological data, the noise and/or motion-induced artifacts caused by the non-facial muscle activity. In an exemplary embodiment of the present disclosure, for generating the set of second error correction values, the temple mounted wearable device 100 may utilize the first trained model to analyze the relevance or contribution of the non-facial muscle activity within the set of first IMU data. In an event that the relevance or contribution of the non-facial muscle activity in the set of first IMU data exceeds a predefined level, such as the second threshold value, the first trained model may generate the set of second error correction values based on the set of non-facial IMU weights.

In an exemplary embodiment of the present disclosure, for generating the set of second error correction values, the first trained model may fetch, from the memory 134 of the temple mounted wearable device 100, a set of historical physiological data including at least a historic set of PPG data and a historic set of IMU data associated with the user 101, wherein the set of historical physiological data may refer to physiological data previously collected during a particular period of time and stored in the memory 134. In such an exemplary embodiment, the first trained model may filter, from the set of historical physiological data, a set of historic non-facial muscle activity IMU data that corresponds to or aligns with the current set of non-facial muscle activity IMU data. Based on the filtered set of historic non-facial muscle activity IMU data, the first trained model may detect one or more movements of one or more body parts associated with the head region of the user 101, such as head rotation, head nodding, or other head-related movements. The first trained model may further identify a set of patterns corresponding to the detected movements of the one or more body parts associated with the head region of the user 101. Thereafter, the first trained model may generate the set of second error correction values based on the identified set of patterns associated with the one or more movements of the one or more body parts associated with the head region of the user 101. Subsequently, the method 700 may include enhancing the set of PPG data based on the set of second error correction values.

Further, the method 700 may include filtering the set of second IMU data from the set of IMU data in an event the identified set of target IMU data includes the set of second IMU data. In an exemplary embodiment, the method 700 may utilize one or more IMU data filtering techniques, such as, but not limited to, a least-mean-square (LMS) filtering technique, a wavelet-based denoising technique, or other signal-processing techniques suitable for filtering the set of second IMU data from the set of IMU data.

It is to be noted that the abovementioned filtering techniques are only exemplary and in no manner intended to limit the scope of the present disclosure. The filtering technique may include any other technique(s) as may be appreciated by the person skilled in the art to implement the present solution.

Further, the method 700 may include determining a set of second IMU attributes associated with the set of second IMU data, wherein the set of second IMU attributes includes a set of second motion attributes associated with the set of second IMU data and a set of second orientation attributes associated with the set of second IMU data. It is to be noted that the term “set of second motion attributes” may refer to data indicative of one or more physical movements of one or more body parts other than the head region of the user 101, such as, but not limited to, acceleration, angular velocity, linear velocity, or rotational rate associated with movements of the hands, shoulders, legs, or other body parts below the head region of the user 101. As used herein the term “set of second orientation attributes” may refer to data indicative of one or more postures or positions of one or more body parts other than the head region of the user 101, such as, but not limited to, the orientation, inclination, or angular displacement of the hands, shoulders, legs, or other body parts below the head region of the user 101.

Further, in an exemplary embodiment of the present disclosure, for determining the set of second IMU attributes, a second trained machine learning (ML) model (also referred to as the second trained model) may be utilized to analyze the set of second IMU data to detect one or more movements of one or more regions or body parts below the head region of the user 101. Further, based on the detected movements, the second trained ML model may determine the set of second IMU attributes associated with the set of second IMU data.

Further, the method 700 may include generating a set of third error correction values associated with the set of second IMU data, based on the set of second IMU attributes. It is to be noted that the set of third error correction values may refer to one or more parameters configured to remove or reduce, from the set of physiological data, the noise and/or motion-induced artifacts caused by the one or more movements of one or more regions or body parts below the head region of the user 101.

Further, in an exemplary embodiment of the present disclosure, for generating the set of third error correction values, the second trained model may fetch, from the memory 134 of the temple mounted wearable device 100, a set of historical physiological data including at least a historic set of PPG data and a historic set of IMU data associated with the user 101, wherein the set of historical physiological data may refer to physiological data previously collected and characterized as the physiological data associated with one or more regions or body parts below the head region of the user 101 during a particular period of time and that may be stored in the memory 134. In such an exemplary embodiment, the second trained model may filter, from the set of historical physiological data, a set of historic second IMU data corresponding to movements of one or more regions or body parts below the head region of the user 101. Based on the filtered set of historic second IMU data, the second trained model may detect one or more movements of the one or more regions or body parts below the head region of the user 101 and may further identify a set of patterns corresponding to the detected movements. Thereafter, the second trained model may generate the set of third error correction values based on the identified set of patterns associated with the one or more movements of the one or more regions or body parts below the head region of the user 101. In an exemplary embodiment, the second trained model may further adjust or refine the set of third error correction values by fetching one or more historic error correction values associated with historic second IMU data that match or align with the current set of second IMU data, thereby enabling adaptive tuning of the error-correction parameters based on previously applied corrections. Thereafter, the method 700 may include enhancing the set of PPG data based on the set of third error correction values.

Next, at step 708, the method 700 includes extracting one or more blood flow indicators associated with the user 101 based on at least one of the set of PPG data and the set of enhanced PPG data, wherein extracting the one or more blood flow indicators is further based on the motion correction status. The one or more blood flow indicators associated with the user 101 may be detected based on the set of PPG data in an event that the motion correction status indicates the negative motion correction status. Similarly, the one or more blood flow indicators associated with the user 101 may be detected based on the set of enhanced PPG data in an event that the motion correction status indicates the positive motion correction status. It is to be noted that the term “one or more blood flow indicators” may refer to one or more attributes/parameters associated with a blood circulation of the user 101, such as, but is not limited to, blood velocity, blood pressure, blood perfusion, etc.

Further, in an exemplary embodiment of the present disclosure, extracting the one or more blood flow indicators may include identifying at least one of one or more temporal features associated with the user 101 based on at least one of the set of PPG data and the set of enhanced PPG data. The one or more temporal features associated with the user 101 may be identified based on the set of enhanced PPG data in an event, the motion correction status indicates the positive motion correction status. Similarly, the one or more temporal features associated with the user 101 may be identified based on the set of PPG data in an event, the motion correction status indicates the negative motion correction status. In such exemplary embodiment, the one or more temporal features may include at least one of a beat length feature, a data periodicity feature, a dicrotic notch feature, a set of phase shift features, a set of derivative features and a set of transient variation features.

It is to be noted that the term “beat length feature” may refer to time-domain attribute indicating the time duration of a single cardiac cycle (e.g., the time duration for completion of a single, complete pulse wave).

Further, it is to be noted that the term “data periodicity feature” may refer to the rhythmic, quasi-periodic repetition of the waveform associated with each cardiac cycle.

Further, it is to be noted that the term “dicrotic notch feature” may refer to a small, downward inflection or “notch” observed on the descending slope of the PPG pulse wave, indicating the end of the systolic phase and the start of the diastolic phase.

Further, it is to be noted that the term “set of phase shift features” may refer to the time delays or temporal misalignment between different points on the PPG waveform or between different synchronized signals.

Further, it is to be noted that the term “set of derivative features” may refer to a features derived from the set of PPG data and/or the set of enhanced PPG data that depict instantaneous change characteristics of the set of PPG data and/or the set of enhanced PPG data on a per-sample basis, including variations in slope or curvature of the waveform associated with the set of PPG data and/or the set of enhanced PPG data.

Furthermore, it is to be noted that the term “transient variation features” may refer to short-lived, dynamic changes in the shape, amplitude, or timing of the PPG pulse wave that may occur over a few cardiac cycles, rather than long-term trends.

Further, in an embodiment of the present disclosure, the method 700 may include determining a set of first indicators associated with the user 101 based on the one or more temporal features. In such exemplary embodiment, the set of first indicators may include at least one of one or more cerebral blood flow (CBF) indicators and one or more brain activity indicators.

It is to be noted that the term “cerebral blood flow indicators” may refer to a specific, non-invasive measurements and derived features within the pulsatile light-absorbance waveform associated with the volume, velocity, or pressure of blood circulating in the brain's microvasculature.

Furthermore, it is to be noted that the term “brain activity indicators” may refer to specific morphological, temporal, and nonlinear features associated with the blood flow signals associated with cognitive, emotional, or neurological states of the user 101.

Further, in an embodiment of the present disclosure, the method 700 may include identifying one or more amplitude features associated with the user 101, based at least one of the set of PPG data and the set of enhanced PPG data, wherein the one or more amplitude features may be identified based on the set of PPG data in an event the negative motion correction status is determined and the one or more amplitude features may be identified based on the set of enhanced PPG data in an event the positive motion correction status is determined. It would be appreciated by the person skilled in the art that the one or more amplitude features associated with the user 101 may be identified based on the set of enhanced PPG data in an event that the motion correction status indicates the positive motion correction status. Similarly, the one or more amplitude features associated with the user 101 may be identified based on the set of PPG data in an event the motion correction status indicates the negative motion correction status. In such exemplary embodiment, the one or more amplitude features may include at least one of a set of data intensity features, a set of peak-to-peak value features, a set of baseline shift features and a set of pulsatility index features.

It is to be noted that the term “set of data intensity features” may refer to one or more attributes associated with light absorbed/reflected by blood vessels, indicating a volume of blood in the blood vessels of the user 101.

Further, it is to be noted that the term “set of peak-to-peak value features” may refer to the vertical distance (amplitude) between the systolic peak (maximum blood volume) and the diastolic trough or notch (minimum blood volume) within a single pulse cycle.

Further, it is to be noted that the term “set of baseline shift features” may refer to one or more changes in non-pulsatile signal level indicating physiological changes (e.g., vascular tone changes) and physical artifacts (e.g., sensor movement) that directly influence the quality and interpretation of heart rate, blood pressure and stress levels of the user 101.

Furthermore, it is to be noted that the term “set of pulsatility index features” may refer to one or more attributes for analysing cardiovascular health (e.g., vascular stiffness, blood volume, and peripheral resistance) based on at least a size of the pulse (amplitude) and an overall blood flow (pulsatility).

Further, in an exemplary embodiment of the present disclosure, the method 700 may include determining a set of second indicators associated with the user 101 based on the one or more amplitude features. In such exemplary embodiment, the set of second indicators includes at least one of a set of cerebral blood volume indicators, a set of brain-wave indicators and a set of physiological response features.

It is to be noted that the term “set of cerebral blood volume indicators” may refer to one or more features indicating volumetric changes in the microvascular bed of the temple region 142 of the user 101.

Further, it is to be noted that the term “set of brain-wave indicators” may refer to one or more morphological features, spectral components, and non-linear dynamics derived from the optical signal indicating cerebral hemodynamic, intracranial pressure (ICP), and autonomic nervous system activity of the user 101.

Furthermore, it is to be noted that the term “set of physiological response features” may refer to one or more objective, quantifiable markers extracted from the optical signal of blood volume changes in blood vessels of the user 101, indicating an autonomic nervous system's control over cardiovascular and respiratory activities, as well as the peripheral vascular health of the user 101.

Further, at step 710, the method 700 includes generating a set of health metrics associated with the user 101, based on the one or more blood flow indicators. It is to be noted that the term “set of health metrics” may refer to data/information indicating one or more values associated with the health of the user 101 collected for a particular duration of time. Further, in an exemplary embodiment of the present disclosure, the set of health metrics may be a personalized set of health metrics (e.g., user-specific health metrics) indicating one or more values associated with a particular/specific user 101. Further, the one or more values in the set of health metrics may be personalized/updated based on the set of physiological data associated with the particular/specific user 101 collected during a particular duration of time, let us say a set of physiological data collected during last 30 days or 40 days or last 08 hours or during 1 PM-8 PM, etc. In an exemplary embodiment of the present disclosure, the first trained model may receive the set of physiological data associated with the user 101 for the particular duration of time and may generate the set of health metrics based on the received set of physiological data.

Further, in an exemplary embodiment of the present disclosure, the method 700 may include generating, the set of health metrics based on one or more of the set of first indicators and the set of second indicators, wherein the set of health metrics includes at least one of one or more heart rate metrics, one or more sleep metric and one or more blood flow metrics. It is to be noted that the term “heart rate metrics” may refer to information/data indicating one or more values associated with the heart/cardiovascular health of the user 101 collected for a particular duration of time. Further, it is to be noted that the term “sleep metric” may refer to information/data indicating one or more values associated with the sleep health/sleep pattern of the user 101 collected for a particular duration of time. Furthermore, it is to be noted that the term “blood flow metric” may refer to information/data indicating one or more values associated with the blood flow of the user 101 collected for a particular duration of time.

It is to be noted that the abovementioned health metrics are only exemplary in nature and in no manner intended to limit the scope of the present disclosure. The set of health metrics may include any other metric(s) as may be appreciated by the person skilled in the art to implement the present solution.

Furthermore, at step 712, the method 700 includes monitoring the set of health attributes associated with the user 101, based on the set of health metrics for determining a health state associated with the user 101. It is to be noted that the term “set of health attributes” may refer to one or more parameters indicating the overall health of the user 101. In an exemplary embodiment of the present disclosure, the set of health attributes may be associated with at least one of one or more heart rate attributes associated with the user 101, one or more sleep attributes associated with the user 101 and one or more blood flow attributes associated with the user 101.

It is to be noted that the term “one or more heart rate attributes” may refer to data indicating one or more features/characteristics associated with the functioning of the heart of the user 101. Further, the one or more heart rate attributes may include, but is not limited to, heartbeat attribute, resting heart rate attribute, heart rate variability (HRV) attribute, maximum heart rate attribute, etc.

It is to be noted that the term “one or more sleep attributes” may refer to data associated with sleep pattern and/or sleep quality of the user 101, such as, but is not limited to, sleep duration attribute, sleep continuity attribute, sleep timing attribute, etc.

It is to be noted that the term “one or more blood flow attributes” may refer to data indicating one or more features/characteristics associated with blood movement in the user 101, such as, but is not limited to, blood flow velocity attribute, Blood Volume Pulse (BVP) attribute, blood pressure attribute, etc.

Further, in an exemplary embodiment of the present disclosure, the method 700 may include receiving in real-time from the temple region 142 of the user 101, a set of target PPG data, wherein the set of target PPG data may include at least one of the set of PPG data and the set of enhanced PPG data. In light of the present disclosure, it would be apparent to a person skilled in the art that the set of target PPG data may include at least the set of PPG data when the negative motion correction status is indicated for at least one of the set of first IMU data and the set of second IMU data. Similarly, the set of target PPG data may include at least the set of enhanced PPG data when the positive motion correction status is indicated for at least one of the set of first IMU data and the set of second IMU data.

Further, the method 700 may include analysing, in real-time, the one or more blood flow indicators associated with the user 101 based on at least the set of real-time target PPG data. It is to be noted that the term “set of real-time target PPG data” may refer to a set of instantaneous PPG data associated with the user 101. For ease of understanding, consider an example in which the user 101 is engaged in light physical activity, such as walking, while wearing the temple mounted wearable device 100 mounted on the temple region 142 of the user 101. As the user 101 moves, the temple mounted wearable device 100 continuously receives real-time PPG data (i.e., instantaneous raw PPG data) from the temple region 142. In an event where the motion correction status is negative, the set of real-time target PPG data corresponds to the instantaneous raw PPG data. Conversely, when the motion correction status is positive, the set of real-time target PPG data may correspond to the enhanced PPG data generated from the instantaneous raw PPG data based on compensating for motion-induced artifacts detected by the at least one IMU sensor 120B. Based on this set of real-time target PPG data, the method 700 may analyze one or more blood-flow indicators of the user 101, such as pulse amplitude variations, systolic and diastolic peak characteristics, or changes in waveform morphology, thereby enabling continuous monitoring a physiological state of the user 101.

Further, the method 700 may include determining a set of real-time health metric values associated with the user 101 based on the one or more real-time blood flow indicators. It is to be noted that the term “set of real-time health metric values” may refer to one or more values associated with the current health data of the user 101. Furthermore, the method 700 may include monitoring the set of health attributes associated with the user 101 based on comparing the set of real-time health metric values and one or more values associated with the set of health metrics to determine the health state of the user 101. In an exemplary embodiment of the present disclosure, the first trained model may be utilised to compare the set of real-time health metric values and one or more values associated with the set of health metrics to identify one or more deviations/changes in the set of health attributes of the user 101.

As used herein, the term “health state of the user” refers to a condition or status associated with the user 101 that is determined based on a comparison between a set of real-time health metric values and one or more values associated with a set of health metrics previously generated for the user 101. The health state of the user 101 reflects one or more instantaneous physiological characteristics derived from real-time blood-flow indicators and other physiological data monitored by the temple mounted wearable device 100. The health state of the user 101 may therefore represent an assessment of whether one or more health attributes of the user 101 exhibit a deviation, change, or variation relative to corresponding baseline or historical health-metric values. In certain embodiments, the health state of the user 101 may be identified by utilising a trained model configured to analyse the set of real-time health metric values and determine whether such values indicate a change in the set of health attributes associated with the user 101.

For ease of understanding, consider an example in which a user A has been wearing the temple mounted wearable device 100 on the temple region 142 for a period of 30 days. During this period, the temple mounted wearable device 100 may continuously receive and store physiological data of the user A, thereby generating a set of historical health data. The historical health data may indicate, for example, that the resting heart rate of user A typically ranges between 60-100 beats per minute. During a current monitoring session, the temple mounted wearable device 100 may receive real-time physiological data indicating a heart rate of 120 beats per minute. The temple mounted wearable device 100 may determine a motion correction status based on detecting one or more real-time movements using at least one of the set of first IMU data and the set of second IMU data. In an event where such real-time movements are detected, the temple mounted wearable device 100 may determine a positive motion correction status and may generate enhanced physiological data by compensating for motion-induced artifacts using the real-time IMU data. The enhanced physiological data may then be used to determine a set of real-time health metric values. Conversely, in an event where real-time movements are not detected, the temple mounted wearable device 100 may determine a negative motion correction status and may directly use the real-time physiological data to determine the set of real-time health metric values. The temple mounted wearable device 100 may thereafter compare the set of real-time health metric values with one or more values associated with the set of health metrics to identify one or more deviations in the health attributes of the user A, i.e., the health state of the user A.

Further, the temple mounted wearable device 100 may be configured to trigger a warning and/or a notification to the user 101 based on the determined health state and one or more predefined warning trigger rules. For example, the temple mounted wearable device 100 may trigger a warning if the heart rate of the user 101 increases by more than a predefined threshold, such as an increase of 20 beats per minute within a duration of 20 minutes. In an exemplary embodiment, the warning and/or notification may be provided to the user 101 in the form of a haptic feedback generated by the temple mounted wearable device 100, a visual or audio notification on a user device associated with the user 101, such as a smartphone, or any other suitable alert mechanism.

In an exemplary embodiment of the present disclosure, the processor 130 may combine the identified the one of one or more temporal features and the one or more amplitude features to determine a Cerebral Blood Flow (CBF) metric of the user 101 in real-time.

In an exemplary embodiment of the present disclosure, the method 700 may further include computing a brain state of the user 101. In such an embodiment of the present disclosure, the processor 130, in conjunction with the sensor unit 120, may enable computation of one or more states of the brain of the user 101. In an implementation, to compute the one or more states of the brain of the user 101, the processor 130 may remove motion-induced artifacts from the set of PPG data, i.e., the noise induced due to the set of IMU data, to generate the set of enhanced PPG data. Further, the processor 130 may isolate delta (0.5 Hz to 4 Hz), theta (4 Hz to 8 Hz), and alpha (8 Hz to 12 Hz) frequency ranges associated with the set of enhanced PPG data. In an embodiment, the isolated frequency range specific data may enable the detection and classification of distinct brainwave patterns, i.e., the set of brain-wave indicators, which are indicative of various cognitive and neurological states. Further, the processor 130 may perform signal processing to enhance the frequencies in the corrected and isolated signals associated with the set of enhanced PPG data. Thereafter, the processor 130 may merge the enhanced corrected and isolated signals to compute the brain state of the user 101. In an embodiment, the brain state may include one or more of, but not limited to, focus, creative, relaxed, stressed, and the like. In an embodiment, the temple mounted wearable device 100 may also provide a true age of the user 101 according to various computations on the brain metrics of the user 101, and the CBF and CBV data calculated. In an embodiment, for the determination of the true age, the processor 130 may calculate a true age score based on a comparison of the CBF metric of the user 101 with a dataset including brain metrics of various users of different age groups.

Further, for ease of understanding, referring to FIG. 8 which illustrates an exemplary processing pipeline implemented by the temple mounted wearable device 100 for monitoring the set of health attributes associated with the user 101, in accordance with the embodiments of the present disclosure. As shown, the pipeline begins with the at least one optical sensor 120A of the sensor unit 120, which is configured to monitor the set of photoplethysmography (PPG) data associated with the user 101. The at least one optical sensor 120A may generate the set of PPG data (also referred to as raw PPG data for ease of understanding) that is received by the processor 130. The processor 130 may implement a motion-correction pipeline that operates on the set of PPG data based on the set of inertial measurement unit (IMU) data received from the at least one IMU sensor 120B of the sensor unit 120. The at least one IMU sensor 120B may monitor the set of IMU data, including at least one of the set of first IMU data associated with the head region of the user 101 and the set of second IMU data associated with the one or more regions below the head region of the user 101. The processor 130 may determine the motion-correction status associated with the set of PPG data based on the set of IMU data, wherein the motion-correction status is one of the positive motion-correction status and the negative motion-correction status. In an event that the positive motion-correction status is determined, the processor 130 may be configured to generate the set of enhanced PPG data based on compensating for noise and motion-induced artifacts induced in the set of PPG data based on the set of IMU data. In an event that the negative motion-correction status is determined, the raw PPG data is utilised as an input by the processor 130 of the temple mounted wearable device 100 for subsequent processing as disclosed by the method 700 above, to monitor the set of health attributes associated with the user 101.

In an embodiment of the present disclosure, once the motion correction status is determined (i.e., whether the motion correction is required or not), and a subsequent motion-correction processing is successfully performed on the raw PPG data in compliance with the method 700 as disclosed above. As illustrated in the FIG. 8 the exemplary processing pipeline includes the extraction of one or more blood-flow indicators based on at least one of the set of PPG data and the set of enhanced PPG data. The extraction includes identifying one or more temporal features and one or more amplitude features associated with the user 101, in accordance with the embodiments described in the specification. Further, in an exemplary implementation of the present disclosure, one or more micro-movements associated with the user 101 may be detected by the temple mounted wearable device 100 based on the extracted indicators. It is to be noted that the term “micro-movements” may refer to a small, brief, and often involuntary physical movement of the user 101, such as chewing, blinking, nodding, or similar subtle actions that momentarily alter the stability or contact characteristics of the temple mounted wearable device 100. These micro-movements may introduce transient variations in the acquired set of physiological data.

Further, in an implementation of the present disclosure, the processor 130 may be configured to generate the set of health metrics associated with the user 101 based on the extracted indicators and/or the detected one or more micro-movements. In such implementation, said set of health metrics includes at least one of the one or more heart rate metrics, the one or more sleep metrics and the one or more blood flow metrics (as discussed above).

Thereafter, the generated set of health metrics may be utilized for monitoring the set of health attributes associated with the user 101, including determining the health state associated with the user 101 based on comparing real-time health-metric values with stored health-metric values. The health state of the user may reflect one or more instantaneous physiological characteristics derived from real-time blood-flow indicators and other physiological data monitored by the temple mounted wearable device 100. The final stage of the pipeline may include generating an output or notification, which may include haptic feedback via the temple mounted wearable device 100 or alerts transmitted to the user device 140, thereby enabling the user 101 to receive real-time updates regarding the monitored set of health attributes.

According to yet another aspect, the present disclosure relates to a non-transitory computer-readable storage medium storing instructions for monitoring the set of health attributes associated with the user 101. The instructions include executable code which, when executed by a processing resource, may cause the processing resource to perform operations including receiving the set of physiological data associated with the user 101, wherein the set of physiological data includes at least one of the set of photoplethysmography (PPG) data and the set of inertial measurement unit (IMU) data. In accordance with an exemplary embodiment, the operations may further include determining the motion correction status associated with the set of PPG data based on the set of IMU data, wherein the motion correction status is one of the positive motion correction status and the negative motion correction status. In accordance with an exemplary embodiment, the operations may further include generating the set of enhanced PPG data based on the set of IMU data in an event that the positive motion correction status is determined. In accordance with an exemplary embodiment, the operations may further include extracting the one or more blood flow indicators associated with the user 101 based on at least one of the set of PPG data and the set of enhanced PPG data, wherein extracting the one or more blood flow indicators is further based on the motion correction status. In accordance with an exemplary embodiment, the operations may further include generating the set of health metrics associated with the user 101 based on the one or more blood flow indicators. In accordance with an exemplary embodiment, the operations may further include monitoring the set of health attributes associated with the user 101 based on the set of health metrics. Thereafter, in accordance with an exemplary embodiment, the operations may further include determining the health state associated with the user 101 based on the set of health attributes.

As evident from the above, the present disclosure provides a technically advanced solution for accurately monitoring the set of health attributes associated with the user 101. The disclosed solution offers several advantages over existing systems positioned on peripheral or above-shoulder locations of the user 101. As disclosed above, the temple mounted wearable device 100 is mounted on the temple region 142 and secured using an adaptive housing with the set of protrusions 110 in combination with the sticking component. This configuration maintains stable and continuous sensor contact in an area with higher blood perfusion, reduced soft-tissue deformation, and significantly lower susceptibility to motion-induced artifacts.

Additionally, such stable placement of the temple mounted wearable device 100 minimizes noise introduced by repetitive limb movement, inconsistent skin coupling, sweat accumulation, and transient contact interruptions, issues that commonly affect prior-known health and fitness monitoring devices worn on the wrist, finger, and forearm. As a result of the lower susceptibility to motion-induced artifacts, the solution of the present disclosure reduces hardware and processing requirements and complexity as compared to prior-known solutions, which require continuous motion-artifact compensation in the captured or monitored signal.

Further, the disclosed solution provides the technical effect of distinguishing between different categories of motion-induced noise, including subtle facial-expression movements, general head motion, and vigorous limb activity. By identifying both the cause and magnitude of noise in the captured signals, the device selectively filters or suppresses noise components and reduces processing operations when noise exceeds a predefined threshold. This targeted noise correction approach improves signal integrity and reduces computational requirements, enabling more efficient and faster real-time processing.

Additionally, the solution described in this disclosure reduces sweat-related noise in the captured signals by incorporating vented structural features, while maintaining consistent sensor coupling without causing discomfort. This approach avoids the fit-related limitations commonly seen in ear-worn or neck-worn devices. Together, these technical effects enable faster and more efficient derivation of health attributes such as heart rate, respiration, sleep patterns, and cerebral blood-flow metrics, using simpler hardware and lower processing complexity, ultimately improving accuracy, stability, and usability for continuous daily monitoring.

While the present invention has been described with reference to certain preferred embodiments and examples thereof, other embodiments, equivalents and modifications are possible and are also encompassed by the scope of the present disclosure.

1. A temple mounted wearable device 100, comprising:
a housing unit 102 comprising at least an aperture 118 positioned to expose a sensor unit 120 to a skin overlying a superficial temporal artery (STA) at a temple region 142 of a user 101; and
a computing unit 104 disposed within the housing unit 102, wherein the computing unit 104 comprises at least:
the sensor unit 120 configured to detect a set of physiological data from the STA; and
a processor 130 connected to at least the sensor unit 120, wherein the processor 130 is configured to process the set of physiological data.

2. The temple mounted wearable device 100 of claim 1, wherein the housing unit 102 comprises a first housing member 106 and a second housing member 108 comprising the aperture 118, and wherein the second housing member 108 is configured to conform to a shape of the temple region 142 of the user 101.

3. The temple mounted wearable device 100 of claim 1, wherein the sensor unit 120 comprises at least one optical sensor 120A and at least one inertial measurement unit (IMU) sensor 120B, and wherein the at least one optical sensor 120A includes one photoplethysmography (PPG) sensor.

4. The temple mounted wearable device 100 of claim 1, wherein the set of physiological data comprises at least a set of photoplethysmography (PPG) data and a set of inertial measurement unit (IMU) data, and wherein the set of IMU data comprises a set of first IMU data associated with a head region of the user 101, and a set of second IMU data associated with one or more regions below the head region of the user 101.

5. The temple mounted wearable device 100 of claim 2, wherein:
the second housing member 108 comprises at least an inner layer 114 and an outer layer 116, wherein the outer layer 116 comprises at least a set of protrusions 110 and a sticking component, and
the sticking component is configured to secure the temple mounted wearable device

100. to the temple region 142 of the user 101.

6. The temple mounted wearable device 100 of claim 5, wherein the set of protrusions 110 and the sticking component define one or more vents.

7. The temple mounted wearable device 100 of claim 2, wherein:
the housing unit 102 further comprises a housing assembly member 112, wherein the housing assembly member 112 uses at least an interlocking technique and an adhesive technique, and
the first housing member 106 and the second housing member 108 define a housing cavity for housing the computing unit 104.

8. The temple mounted wearable device 100 of claim 1 further comprising an insulation layer 122 coupled to at least the computing unit 104, wherein the insulation layer 122 is configured to insulate the user 101 from a set of electromagnetic (EM) waves associated with the temple mounted wearable device 100.

9. The temple mounted wearable device 100 of claim 1, wherein to process the set of physiological data, the processor 130 is configured to:
receive from the sensor unit 120 the set of physiological data associated with the user 101;
determine a motion correction status associated with the set of PPG data based on the set of IMU data, wherein the motion correction status is one of a positive motion correction status and a negative motion correction status;
generate a set of enhanced PPG data based on the set of IMU data in an event, the positive motion correction status is determined;
extract one or more blood flow indicators associated with the user 101 based on at least one of the set of PPG data and the set of enhanced PPG data, wherein extracting the one or more blood flow indicators is further based on the motion correction status;
generate a set of health metrics associated with the user 101 based on the one or more blood flow indicators;
monitor the set of health attributes associated with the user 101 based on the set of health metrics; and
determine a health state associated with the user 101 based on the set of health attributes.

10. The temple mounted wearable device 100 of claim 9, wherein the set of health attributes is associated with at least one of one or more heart rate attributes, one or more sleep attributes, one or more respiratory attributes, one or more brain attributes and one or more blood flow attributes.

11. A method for monitoring a set of health attributes associated with a user 101, the method comprising:
receiving a set of physiological data associated with the user 101, wherein the set of physiological data comprises at least a set of photoplethysmography (PPG) data and a set of inertial measurement unit (IMU) data;
determining a motion correction status associated with the set of PPG data based on the set of IMU data, wherein the motion correction status indicates one of a positive motion correction status and a negative motion correction status;
generating a set of enhanced PPG data based on the set of IMU data in an event the positive motion correction status is determined;
extracting one or more blood flow indicators associated with the user 101 based on at least one of the set of PPG data and the set of enhanced PPG data;
generating a set of health metrics associated with the user 101 based on the one or more blood flow indicators; and
determining a health state associated with the user 101 based on monitoring the set of health attributes.

12. The method of claim 11, wherein the set of physiological data is detected from a superficial temporal artery (STA) associated with a temple region 142 of the user 101.

13. The method of claim 11 further comprising:
determining a temporal alignment status associated with the set of IMU data and the set of PPG data, wherein the temporal alignment status is one of a positive temporal alignment status and a negative temporal alignment status;
determining in the set of physiological data, a set of IMU weights associated with the set of IMU data in an event, the positive temporal alignment status is determined; and
determining the motion correction status associated with the set of PPG data based on the set of IMU weights.

14. The method of claim 13, wherein:
the positive motion correction status associated with the set of PPG data is determined in an event one or more weight values in the set of IMU weights is one of equal to a first predefined threshold value and above the first predefined threshold value, and
the negative motion correction status associated with the set of PPG data is determined in an event one or more weight values in the set of IMU weights is one of below the first predefined threshold value.

15. The method of claim 13, wherein:
the positive temporal alignment status associated with the set of IMU data and the set of PPG data is determined in an event a set of IMU data time-period values associated with the set of IMU data is equal to a set of PPG data time-period values associated with the set of PPG data, and
the negative temporal alignment status associated with the set of IMU data and the set of PPG data is determined in an event the set of IMU data time-period values associated with the set of IMU data differs from the set of PPG data time-period values associated with the set of PPG data.

16. The method of claim 11, wherein the set of IMU data comprises one or more of a set of first IMU data associated with a head region of the user 101, and a set of second IMU data associated with one or more regions below the head region of the user 101.

17. The method of claim 16 further comprising:
determining in the set of IMU data, at least one of a set of first IMU weight associated with the set of first IMU data and a set of second IMU weight associated with the set of second IMU data in an event the positive motion correction status is determined;
identifying from the set of IMU data, a set of target IMU data based on the set of first IMU weight and the set of second IMU weight and a second predefined threshold value; and
generating the set of enhanced PPG data based on the set of target IMU data.

18. The method of claim 17, wherein:
the set of first IMU data is identified as the set of target IMU data in an event a value of the set of first IMU weight in the set of IMU data is one of equal to the second predefined threshold value and above the second predefined threshold value; and
the set of second IMU data is identified as the set of target IMU data in an event a value of the set of second IMU weight in the set of IMU data is one of equal to the second predefined threshold value and above the second predefined threshold value.

19. The method of claim 17 further comprising:
filtering the set of first IMU data from the set of IMU data in an event the identified set of target IMU data comprises the set of first IMU data;
determining a set of first IMU attributes associated with the set of first IMU data, wherein the set of first IMU attributes comprises at least one of a set of first motion attributes and a set of first orientation attributes;
identifying a set of first user activity types associated with the set of first IMU data based on the set of first IMU attributes, wherein the set of first user activity types comprises at least one of a facial muscle activity and a non-facial muscle activity;
filtering from the set of first IMU data, a set of facial muscle activity IMU data and a set of non-facial muscle activity IMU data based on the set of first user activity types; and
generating the set of enhanced PPG data based on at least one of the set of facial muscle activity IMU data and the set of non-facial muscle activity IMU data.

20. The method of claim 19, wherein the set of facial muscle activity IMU data is associated with one or more facial muscles of the user 101, and wherein the set of non-facial muscle activity IMU data is associated with one or more body part movement associated with the head region of the user 101.

21. The method of claim 19 further comprising:
fetching a first set of error correction values from a set of predefined error correction values based on the set of facial muscle activity IMU data in an event the identified set of first user activity types comprises the facial muscle activity; and
generating the set of enhanced PPG data based on the first set of error correction values.

22. The method of claim 19 further comprising:
determining a set of non-facial IMU attributes associated with the non-facial muscle activity IMU data in an event the identified set of first user activity types comprises the non-facial muscle activity;
determining in the set of first IMU data, a set of non-facial IMU weights associated with the set of non-facial muscle activity IMU attributes;
generating a set of second error correction values associated the non-facial muscle activity based on the set of non-facial IMU weights; and
enhancing the set of PPG data based on the set of second error correction values.

23. The method of claim 17 further comprising:
filtering the set of second IMU data from the set of IMU data in an event the identified set of target IMU data comprises the set of second IMU data;
determining a set of second IMU attributes associated with the set of second IMU data, wherein the set of second IMU attributes comprises a set of second motion attributes associated with the set of second IMU data and a set of second orientation attributes associated with the set of second IMU data;
generating a set of third error correction values associated with the set of second IMU data based on the set of second IMU attributes; and
enhancing the set of PPG data based on the set of third error correction values.

24. The method of claim 11, wherein extracting the one or more blood flow indicators further comprises:
identifying at least one of one or more temporal features associated with the user 101 based at least one of the set of PPG data and the set of enhanced PPG data, wherein the one or more temporal features are identified based on the set of PPG data in an event the negative motion correction status is determined and the one or more temporal features are identified based on the set of enhanced PPG data in an event the positive motion correction status is determined;
determining a set of first indicators associated with the user 101 based on the one or more temporal features;
identifying one or more amplitude features associated with the user 101 based on at least one of the set of PPG data and the set of enhanced PPG data, wherein the one or more amplitude features are identified based on the set of PPG data in an event the negative motion correction status is determined and the one or more amplitude features are identified based on the set of enhanced PPG data in an event the positive motion correction status is determined; and
determining a set of second indicators associated with the user 101 based on the one or more amplitude features.

25. The method of claim 24, further comprises generating, the set of health metrics based on one or more of the set of first indicators and the set of second indicators, wherein the set of health metrics comprises at least one of one or more heart rate metrics, one or more sleep metrics and one or more blood flow metrics.

26. The method of claim 24, wherein:
the one or more temporal features comprises at least one of a beat length feature, a data periodicity feature, a dicrotic notch feature, a set of phase shift features, a set of derivative features, and a set of transient variation features,
the set of first indicators comprises at least one of one or more cerebral blood flow (CBF) indicators and one or more brain activity indicators,
the one or more amplitude features comprises at least one of a set of data intensity features, a set of peak-to-peak value features, a set of baseline shift features and a set of pulsatility index features, and
the set of second indicators comprises at least one of a set of cerebral blood volume (CBV) indicators, a set of brain-wave indicators and a set of physiological response features.

27. The method of claim 11, wherein the set of health attributes is associated with at least one of one or more heart rate attributes associated with the user 101, one or more sleep attributes associated with the user 101 and one or more blood flow attributes associated with the user 101.

28. The method of claim 12 further comprising:
receiving in real-time from the temple region 142 of the user 101, a set of target PPG data, wherein the set of target PPG data comprises at least one of the set of PPG data and the set of enhanced PPG data;
extracting in real-time, the one or more blood flow indicators associated with the user 101 based on at least the set of real-time target PPG data;
determining a set of real-time health metric values associated with the user 101 based on one or more real-time blood flow indicators; and
monitoring the set of health attributes associated with the user 101 based on comparing the set of real-time health metric values and one or more values associated with the set of health metrics to determine the health state of the user 101.

29. A system for monitoring a set of health attributes of a user 101, the system comprising:
a temple mounted wearable device 100 configured to be positioned at a temple region 142 of the user 101, the temple mounted wearable device 100 comprising:
a sensor unit 120 comprising at least one optical sensor 120A and at least one inertial measurement unit (IMU) sensor 120B, wherein the at least one optical sensor 120A is configured to detect a set of photoplethysmography (PPG) data from a superficial temporal artery (STA) at the temple region 142 and the at least one IMU sensor 120B is configured to detect a set of inertial measurement unit (IMU) data associated with the user 101; and
a processor 130 configured to perform a real-time motion artifact correction on the set of PPG data using the set of IMU data, and to derive one or more blood flow indicators from at least one of the set of PPG data and set of enhanced PPG data;
a user device 140 communicatively coupled to the temple mounted wearable device 100 and is configured to receive the one or more blood flow indicators; and
a server 150 communicatively coupled to the user device 140 and configured to generate a set of health metrics associated with the user based on the one or more blood flow indicators, and to determine a health state of the user based on the set of health metrics.

30. A non-transitory computer readable storage medium storing instruction for monitoring a set of health attributes associated with a user 101, the instructions comprising executable code which when executed by a processor, causes the processor to perform operations of claim 11.
